(19) United States
(12) Patent Application Publication
(10) Pub. No.: US 2026/0308399 A1
(43) Pub. Date: Oct. 8, 2026

(54) SUPERHYDROPHOBIC ANTIFOULING COATING COMPOSITIONS FOR RELEASING NITRIC OXIDE AND APPLICATIONS THEREOF

(71) Applicant: University of Georgia Research Foundation, Inc., Athens, GA (US)

(72) Inventors: Hitesh Handa, Athens, GA (US); Lori Marie Estes Bright, Ball Ground, GA (US); Ekrem Ozkan, Ithaca, NY (US)

(21) Appl. No.: 19/480,400

(22) PCT Filed: May 7, 2024
(86) PCT No.: PCT/US2024/028081

§ 371 Date: Oct. 31, 2025

Related U.S. Application Data
(60) Provisional application No. 63/501,192, filed on May 10, 2023.

(51) Int. Cl. C09D 7/62; A61L 2/232; A61L 27/34; A61L 103/15; B05D 1/02; B05D 1/18; B05D 3/02; B05D 7/00; C09D 7/20; C09D 7/40; C09D 183/04

(52) U.S. Cl. CPC C09D 7/62; A61L 2/232; A61L 27/34; B05D 1/02; B05D 1/18; B05D 3/0254; B05D 7/544; C09D 7/20; C09D 7/67; C09D 183/04; A61L 2103/15; B05D 2518/10

(57) ABSTRACT

Described herein are hydrophobic coating compositions having unique physical properties. The coating compositions are composed of silicon oxide nanoparticles and silicon nanoparticles modified with a perfluorolkylsiloxane. The coating compositions can be applied to any article or any surface of an article where it is desirable to reduce or prevent the growth of bacteria. The articles coated with the hydrophobic coating compositions are subsequently treated with a nitric oxide releasing compound that can prevent the growth of bacteria and adhesion of platelets on the article.

### CROSS-REFERENCE TO RELATED APPLICATIONS

This application claims the benefit of and priority to U.S. Provisional Patent Application No. 63/501,192, filed on May 10, 2023, the contents of which are incorporated by reference herein in their entireties.

### STATEMENT REGARDING FEDERALLY SPONSORED RESEARCH OR DEVELOPMENT

This invention was made with government support under R01 HL134899 awarded by the National Institutes of Health. The government has certain rights in the invention.

### BACKGROUND

Blood-contacting biomedical devices are indispensable in healthcare settings, delivering lifesaving therapies to millions of patients every year [1-3]. However, the performance of existing medical devices is frequently impeded by bacterial contamination and thrombosis that lead to device failure and a substantial increase in prolonged hospital stays, healthcare costs, patient morbidity, and mortality [4,5]. The implantation of medical devices often results in difficult-to-treat chronic infections resulting from the attachment and colonization of biofilm-growing and drug-resistant bacteria [6,7]. The CDC reported that on any given day, 1 in 31 patients has a hospital-acquired infection, leading to horrifying financial and medical consequences [8]. Beyond the issue of bacteria-associated infections, indwelling medical devices are prone to rapid occlusion due to thrombosis as a result of the adherence and activation of platelets in flowing blood to the device surfaces, which may result in thromboembolic complications and device dysfunction [9-12]. Systemic antibiotic and anticoagulant treatments are often utilized to avoid these problems; however, they have the disadvantages of promoting bacterial antibiotic resistance and inducing fatal complications such as thrombocytopenia, ischemia, and bleeding [12-14].

Hence, novel approaches to develop biomaterials with synergistically antimicrobial and anti-thrombogenic functionalities for medical device applications are sorely needed.

### SUMMARY

Described herein are hydrophobic coating compositions having unique physical properties. The coating compositions are composed of silicon oxide nanoparticles and silicon nanoparticles modified with a perfluorolkylsiloxane. The coating compositions can be applied to any article or any surface of an article where it is desirable to reduce or prevent the growth of bacteria. The articles coated with the hydrophobic coating compositions are subsequently treated with a nitric oxide releasing compound that can prevent the growth of bacteria and adhesion of platelets on the article.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1D show (a) the schematic illustration of the fabrication process of SH—SiAgNO; (b) SEM images of various SR surfaces, where the inset images are photographs of corresponding SR samples; (c) the elemental compositions; and (d) XRD pattern of SH—SiAgNO, where cale bars correspond to 50 μm.

FIGS. 2A-2D show (a) CA, CAH, and SA of water droplets on various SR surfaces; (b) photographs of water droplets on corresponding SR surfaces; (c) SR; and (d) SH—SiAgNO surfaces to water.

FIGS. 3A-3F show (a) decomposition of the SNAP molecule occurs in the presence of heat, light, and metal ions and leads to the production of two moles of NO per two moles of RSNO/SNAP; (b) NO release over 7 days remained in the physiologically relevant range for all sample types; (c) instantaneous NO release from a representative sample for each film type on day 0; (d) SNAP leaching over 7 d revealed a decrease in leaching for superhydrophobic samples (SH—SiNO and SH—SiAgNO); (e) Ag+ release from SH—SiAg and SH—SiAgNO samples when incubated in PBS for 24 h at 37° C., mirroring the biological studies; and (f) H₂O₂ sterilization of SH—SiAgNO samples revealed no significant changes in SNAP loading, and the water contact angle remained >150°, confirming maintained superhydrophobicity. Statistical significance is denoted as ** (p<0.01) and *** (p<0.001).

FIGS. 4A-4B show antibacterial assay against (a) E. coli revealed a 3-log reduction in viable CFUs for SH—SiAgNO films compared to control SR and (b) S. aureus bacterial assay showed a 2-log reduction compared to control SR. Data are represented as mean±standard deviation and statistical significance is denoted as * (p<0.05), ** (p<0.01), *** (p<0.001), and **** (p<0.0001).

FIGS. 5A-5C show (a) the density of adhered platelets on the surfaces of SR, SH—SiAg, NO, and SH—SiAgNO; (b) Hemolysis rate of various samples; and (c) photographs of microcentrifuge tubes containing the supernatants obtained from the corresponding samples after incubating in porcine blood, demonstrating the lack of lysed blood cells. Statistical significance is denoted as **** (p<0.0001).

FIGS. 6A-6B show cytocompatibility of SH—SiAgNO surfaces (a) percent viability of mouse fibroblasts after exposure to leachates for 24 h. Values expressed are mean±standard deviation and (b) Fluorescent images showing live and dead mouse fibroblast cells after 24 hours of exposure to leachates. Scale bars correspond to 200 μm. Data are represented as mean±standard deviation and statistical significance is denoted as * (p<0.05), ** (p<0.01), *** (p<0.001), and **** (p<0.0001).

FIG. 7 shows SEM and EDS mapping images of the untreated and treated powders. Scale bars correspond to 2.5 μm.

FIGS. 8A-8B show FTIR analysis of (a) silicon dioxide and (b) silicon dioxide+silver nanoparticles treated with FAS. Quantitative KBr sampling (0.005 wt. %) with peak subtraction demonstrated nanoparticle functionalization with FAS, as indicated by the emergence of C—F linkages.

FIG. 9 shows digital photographs of untreated hydrophilic NPs dispersed in water and FAS-treated hydrophobic NPs floated on water after ultrasonic irradiation for 15 min. The third vial is clear glass but was contaminated with Ag NPs following the ultrasonic irradiation step and therefore appears amber.

FIG. 10 shows photographs of vials containing solutions prepared from the SH SiO₂ NPs, SH SiO₂+Ag NPS, and SNAP, from left to right.

FIG. 11 shows Ag⁺ standard curve to quantify silver leaching from the polymer samples.

FIG. 12 shows the cross-sectional photograph of the SHAgNO sample. The sample dimension is 1.27 cm in diameter and 0.19 cm in height.

FIG. 13 shows EDS surface elemental mapping of all film sample types. Scale bars correspond to 50 μm.

FIG. 14 shows an image of the SHAgNO sample immersed in water, demonstrating the air plastron phenomenon owing to the reflection of entrapped air between the rough structures and water.

FIG. 15 shows SNAP loaded into each sample type normalized to film mass (mg). n=5

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” include, but are not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “alkyl group” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer.

The term “perfluoroalkyl group” refers to an alkyl group as defined herein where two or more hydrogen atoms on the alkyl group are substituted with a fluorine atom. In one aspect, all of the hydrogen atoms on the alkyl group are substituted with a fluorine atom.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms (e.g., bacteria, fungi, protozoans, algae, and the like). A substance having an antimicrobial characteristic can kill the microorganism and/or prevent or substantially prevent the growth or reproduction of the microorganism.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila branhamella, Borrelia, Bordetella, Brachyspira, Brevibacillus, Brevibacterium, Brevundimonas, Brucella, Burkholderia, Buttiauxella, butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydia trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Coating Compositions

Described herein are hydrophobic coating compositions having unique mechanical and physical properties. The coating compositions are composed of silicon oxide nanoparticles and silicon nanoparticles modified with a perfluorolkylsiloxane, which are referred to herein as hydrophobically-modified nanoparticles. The coating compositions can be applied to any article or any surface of an article where it is desirable to reduce or prevent the growth of bacteria. The articles coated with the hydrophobic coating compositions are subsequently treated with a nitric oxide releasing compound that can prevent the growth of bacteria and adhesion of platelets on the article. Not wishing to be bound by theory, the release of nitric oxide acts as an antimicrobial agent and platelet inhibitor, while the superhydrophobic components of the coating composition prevent non-specific biofouling and further improves antimicrobial activity.

The coating compositions described herein include hydrophobically-modified nanoparticles, where a perfluorolkylsiloxane is covalently bonded to each of the nanoparticles. In one aspect, the hydrophobically-modified nanoparticles described herein can be made by admixing silicon oxide nanoparticles and silver nanoparticles with a perfluorolkylsiloxane in an organic solvent. The components can be sequentially added to the organic solvent or, in the alternative, the components can be added concurrently to the organic solvent. The duration of mixing of the components can vary as well as the temperature. In one aspect, the components are mixed at from 20° C. to 30° C., or at room temperature. Upon completion of the reaction, the solvent can be removed to produce the hydrophobically-modified nanoparticles on dry form for future use.

Upon mixing the perfluorolkylsiloxane with the silicon oxide nanoparticles and the silver nanoparticles, the perfluorolkylsiloxane can form a covalent bond with the silicon oxide nanoparticles and silver nanoparticles. In one aspect, the silicon oxide nanoparticles and the silver nanoparticles react with the siloxane group of the perfluorolkylsiloxane to produce new Si—Si and Si—Ag bonds. The Examples provide non-limiting procedures for making the hydrophobically-modified nanoparticles present on the coating compositions describe herein.

In one aspect, the silicon oxide nanoparticles can have an average particle size of about 50 nm to about 50 nm, or about 5 nm, 10 nm, 15 nm, 20 nm, 25 nm, 30 nm, 35 nm, 40 nm, 45 nm, or 50 nm, where any value can be a lower and upper endpoint of a range (e.g., 40 nm to 50 nm).

In another aspect, the silver nanoparticles can have an average particle size of about 5 nm to about 100 nm, or about 5 mm, 10 mm, 15 mm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, or 100 nm, where any value can be a lower and upper endpoint of a range (e.g., 40 nm to 50 nm).

The perfluorolkylsiloxane is a compound having a perfluoroalkyl group covalently bonded to a siloxane group. In one aspect, the perfluorolkylsiloxane can have the formula R²—Si(OR¹)₃, wherein R¹ is a substituted or unsubstituted C₁-C₂₀ alkyl group, and R² is a C₁-C₂₀ perfluoroalkyl group. In other aspects, R¹ can be a C₁ to C₄ alkyl group. R² can be a C₁ to C₁₀ perfluoroalkyl group. In one aspect, each R¹ can be methyl or ethyl, and R² can be a C₈ perfluoroalkyl group. In some embodiments, the coating composition can include about 0.1% to about 2% perfluorolkylsiloxane by weight, or about 0.1%, 0.2%, 0.3%, 0.4%, 0.5%, 0.6%, 0.7%, 0.8%, 0.9%, 1.1%, 1.2%, 1.3%, 1.4%, 1.5%, 1.6%, 1.7%, 1.8%, 1.9%, or 2.0%, where any value can be a lower and upper endpoint of a range (e.g., 0.3% to 1.5%).

In one aspect, the organic solvent used to produce the hydrophobically-modified nanoparticles can include an alcohol. In one aspect, the alcohol can be a C₁ to C₁₀ alcohol. In another aspect, the organic solvent can include such as methanol, ethanol, propanol, isopropanol, butanol, or any combination thereof. In another aspect, the organic solvent can be a hydrocarbon such as, for example, hexane.

The hydrophobically-modified nanoparticles can subsequently be formulated with other components to produce a coating compositions that can readily be applied to a surface of an article. In one aspect, the hydrophobically-modified nanoparticles can be mixed in an organic solvent that can readily be removed after application to the coating composition to the article. In one aspect, the organic solvent is an alcohol or a hydrocarbon. In another aspect, the organic solvent is a C₁ to C₁₀ hydrocarbon (e.g., hexane).

The amount of the hydrophobically-modified nanoparticles present in the coating compositions described herein can vary. In one aspect, the coating compositions described herein can include about 1% to about 20% hydrophobically-modified silicon oxide nanoparticles by weight, or about 1%, 2%, 3%, 4%, 5%, 6%, 7%, 8%, 9%, 10%, 11%, 12%, 13%, 14%, 15%, 16%, 17%, 18%, 19%, or 20%, where any value can be a lower and upper endpoint of a range (e.g., 3% to 7%). In another aspect, the coating compositions described herein can include about 1% to about 20% hydrophobically-modified silver oxide nanoparticles by weight, or about 1%, 2%, 3%, 4%, 5%, 6%, 7%, 8%, 9%, 10%, 11%, 12%, 13%, 14%, 15%, 16%, 17%, 18%, 19%, or 20%, where any value can be a lower and upper endpoint of a range (e.g., 3% to 7%).

The relative amount of the hydrophobically-modified silicon oxide nanoparticles and silver nanoparticles can vary as well. In one aspect, the weight ratio of the hydrophobically-modified silicon oxide nanoparticles to the hydrophobically-modified silver nanoparticles is from 0.1 to 5 to 5:0.1, or is 0.1 to 5, 0.5 to 4, 1 to 3, or 1.5 to 2.5.

In addition to the hydrophobically-modified nanoparticles, the coating compositions can include additional components that can facilitate the bonding or adhesion of the hydrophobically-modified nanoparticles to a surface of an article. In one aspect, the coating composition can include an elastomeric polymer. Examples of elastomeric polymers include, but are not limited to, one or more of a copolyester elastomer, a polyether block amide elastomer, a polyurethane elastomer, a polyolefin based-copolymer elastomer, a styrenic copolymer elastomer, an ionomer elastomer, or any combination thereof. In another aspect, the elastomeric polymer is silicone elastomer such as, for example, SYLGARD™ 184 manufactured by Dow Chemical Co.

The selection of the elastomeric polymer can vary depending upon the chemical composition of the article to be coated. For example, if the article to be coated is composed of silicone rubber, then the elastomeric polymer can be a silicone elastomer. In certain aspects, elastomer polymer can include a curing agent to facilitate bonding or adhesion of the coating composition once applied to the article and subsequently cured.

The amount of elastomeric polymer that can be used in combination with the hydrophobically-modified nanoparticles can vary. In one aspect, the coating compositions described herein can include about 25% to about 75% by weight elastomeric polymer, or about 25%, 30%, 35%, 40%, 45%, 50%, 55%, 60%, 65%, 70%, or 75%, where any value can be a lower and upper endpoint of a range (e.g., 40% to 60%). In another aspect, the weight ratio of the silicon oxide nanoparticles and the silver nanoparticles to the elastomeric polymer is from 0.1 to 5 to 5:0.1, or 0.1 to 5, 0.5 to 3, 1 to 2, 1.5 to 2, 1 to 1, or 2 to 1.

When the coating composition includes an elastomeric polymer, the polymer and the hydrophobically-modified nanoparticles can be mixed together using techniques known in the art. In certain aspects, an organic solvent can be added to the mixture to facilitate mixing. In one aspect, the solvent is a C₁ to C₁₀ hydrocarbon such as, for example, hexane. The Examples provide non-limiting examples for preparing coating compositions described herein.

Described herein are articles coated with the compositions described herein. In one aspect, the coated article is produced by
(a) applying the coating composition described herein to at least one surface of the article;
(b) removing the solvent from the coating composition to produce a first coated article; and
(c) applying a nitric oxide releasing compound to the first coated article to produce a second coated article.

The coating composition can be applied to the article using techniques known in the art such as, for example, dipping or spraying. In one aspect, a single coating can be applied to the article. In other aspects, multiple coatings can be sequentially applied to the article. After the coating composition is applied to the article, the coating can be heated to remove any organic solvent if a solvent is used. In one aspect, the organic solvent can be removed by evaporation. In one aspect, the organic solvent can be removed be heating the coated article at a temperature of from about 80° C. to about 120° C. In one aspect, heating is sufficient to remove at least 80%, at least 85%, at least 90%, at least 95%, at least 99%, or 100% of the organic solvent. Upon heating in air, some of the silver nanoparticles may oxidize. In one aspect, the coating composition can also include Ag₂O after removal of the organic solvent. In certain aspects, when a curing agent is present in the coating composition with the elastomeric polymer, the heating step can also induce curing of the polymer. The Examples provide non-limiting examples for applying coating compositions described herein to an article.

After the coating composition, as been applied to the article, a nitric oxide releasing compound is applied to the coating on the article. The nitric oxide releasing compound can be prepared as a solution and subsequently applied to the article using techniques known in the art such as, for example, dipping or spraying. In one aspect, a single coating of the nitric oxide releasing compound can be applied to the coated article. In other aspects, multiple coatings of the nitric oxide releasing compound can be sequentially applied to the coated article. After the nitric oxide releasing compound is applied to the article, the article can be rinsed with a solvent to remove any excess nitric oxide releasing compound. The Examples provide non-limiting examples for applying the nitric oxide releasing compound to articles coated with a coating composition described herein.

The nitric oxide releasing compound is a compound that possesses one or more nitric oxide groups, wherein nitric oxide is subsequently released from the compound. In one aspect, the nitric oxide releasing compound is a S-nitrosothiol compound. In another aspect, the nitric oxide compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

In one aspect, the amount of the nitric oxide releasing compound applied to the coated article is from about 0.1 mg/ml of the composition to about 50 mg/ml of the composition. In another aspect, the amount of the nitric oxide releasing compound is 0.1 mg/ml, 0.5 mg/ml, 1.0 mg/ml, 5.0 mg/ml, 10.0 mg/ml, 15.0 mg/ml, 20.0 mg/ml, 25.0 mg/ml, 30.0 mg/ml, 35.0 mg/ml, 40.0 mg/ml, 45.0 mg/ml, or 50.0 mg/ml, where any value can be a lower and upper endpoint of a range (e.g., 20.0 mg/ml to 35.0 mg/ml).

### Applications of Coating Compositions

The article to be coated can be any article or surface where it is desirable to reduce or prevent biofouling (e.g. growth of bacteria, adhesion of platelets, adhesion of fibrinogen), and products including the coating compositions. Biofilm and thrombus formation on surfaces results in significant morbidity and mortality worldwide, which highlights the importance of the development of efficacious fouling-prevention approaches. Provided herein are highly robust and superhydrophobic coatings with outstanding multi-liquid repellency, bactericidal performance, and extremely low bacterial and blood adhesion, which can be fabricated by a simple two-step dip-coating method.

In one aspect, the coating compositions described herein are useful in applications where it is desirable to reduce or prevent biofouling. Implantable medical devices are a leading cause of infection such as nosocomial infections. Implantable devices coated with or constructed of the compositions described herein can reduce or prevent biofouling in a subject when the device is introduced into the subject. In one aspect, the compositions described herein can reduce or prevent bacterial growth on a surface of an implantable device. In another aspect, the compositions described herein can reduce or prevent biofilm formation on a surface of an implantable device. In another aspect, the compositions described herein can reduce or prevent fibrinogen formation on a surface of an implantable device.

In one, the implantable device is a urinary catheter, artificial heart valve, a vascular catheter, a graft, or a stent. In other aspects, the device is intended to contact human blood or tissue. In one aspect, the device is a hemodialysis device or a component thereof. The coating compositions described herein are biocompatible (e.g., with fibroblast cells), which makes them useful in implantable medical devices.

The compositions described herein can be incorporated into devices in a number of different ways. In one aspect, the devices can be coated with the compositions described herein. The coating thickness can vary as well depending upon the device and application selected. In one aspect, the coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In other aspects, the compositions described herein can be used to fabricate a device. For example, when the device is composed of rubber or includes a rubber component, the rubber can be prepared such that the composition described herein is dispersed throughout the rubber to produce a nitric oxide releasing rubber. Once the nitric oxide releasing rubber has been produced, it can be used to produce devices (e.g., medical implantable devices).

In another aspect, the coating compositions described herein are useful in applications where it is desirable to reduce or prevent biofouling on polymeric medical grade materials (e.g. silicone, PVC, PU). In other aspects, the coating compositions described herein are useful in applications where it is desirable to reduce or prevent biofouling on metals (e.g. steel, titanium). In other aspects, the coating compositions described herein are useful in applications where it is desirable to reduce or prevent biofouling on hospital touch surfaces (e.g. bed rails, bed frames, and handles). In other aspects, the coating compositions described herein are useful in applications where it is desirable to reduce or prevent biofouling on an automobile surfaces boat hull, or aircraft.

In one aspect, the coating compositions described herein can prevent the growth of bacteria on an article, in which the method includes applying the coating composition as above to at least one surface of the article. The coated article can prevent about at least 80%, at least 85%, at least 90%, at least 95%, at least 99%, or 100% of the growth of bacteria when compared to an uncoated article.

In one aspect, the coating compositions described herein can prevent the adhesion of fibrinogen on an article, in which the method includes applying the coating composition as above to at least one surface of the article. The coated article can prevent about at least 80%, at least 85%, at least 90%, at least 95%, at least 99%, or 100% of the adhesion of fibrinogen when compared to an uncoated article.

In one aspect, the coating compositions described herein can prevent the adhesion of platelets on an article, in which the method includes applying the coating composition as above to at least one surface of the article. The coated article can prevent about at least 80%, at least 85%, at least 90%, at least 95%, at least 99%, or 100% of the adhesion of platelets when compared to an uncoated article.

The coating compositions described herein are very hydrophobic (i.e., superhydrophobic). The degree of hydrophobicity can be measured by the contact angle of the coating. In one aspect, the coating and coated article has a water contact angle of from about 75 degrees to about 125 degrees, or about 75 degrees, 85 degrees, 95 degrees, 105 degrees, 115 degrees, or 125 degrees, where any value can be a lower and upper endpoint of a range (e.g., 85 degrees to 115 degrees). In another aspect, the coating and coated article can have an advancing contact angle of from about 150 degrees to about 250 degrees, or about 150 degrees, 160 degrees, 170 degrees, 180 degrees, 190 degrees, 200 degrees, 210 degrees, 220 degrees, 230 degrees, 240 degrees, or 250 degrees, where any value can be a lower and upper endpoint of a range (e.g., 170 degrees to 210 degrees). In another aspect, the coating and coated article can have a contact angle hysteresis of from about 0.1 degrees to about 5 degrees, or about 0.1 degrees, 0.5 degrees, 1.0 degrees, 1.5 degrees, 2.0 degrees, 2.5 degrees, 3.0 degrees, 3.5 degrees, 4.0 degrees, 4.5 degrees, or 5.0 degrees, where any value can be a lower and upper endpoint of a range (e.g., 1.5 degrees to 4.0 degrees). In another aspect, the coating and coated article can have a water sliding angle of from about 0.1 degrees to about 5 degrees, or about 0.1 degrees, 0.5 degrees, 1.0 degrees, 1.5 degrees, 2.0 degrees, 2.5 degrees, 3.0 degrees, 3.5 degrees, 4.0 degrees, 4.5 degrees, or 5.0 degrees, where any value can be a lower and upper endpoint of a range (e.g., 1.5 degrees to 4.0 degrees).

In another aspect, the coating and coated article can have a static water contact angle of greater than 150 degrees. With the high degree of hydrophobicity, the coating and coted articles described herein can repel various liquids including, but not limited to, water, milk, coffee, juice, and blood.

In addition to be hydrophobic, the coating compositions described herein can provide sustained release of nitric oxide. In one aspect, the coatings and coated articles described herein can release nitric oxide for at least 7 days. In another aspect, the rate of release of nitric oxide from the coatings and coated articles described herein is 1 to 5 times less than the same article coated with the coating composition in the absence of silver nanoparticles. In another aspect, the rate of release of nitric oxide from the coatings and coated articles described herein is 1 to 5 times less than the same article coated with only the nitric oxide releasing compound.

### Aspects

Aspect 1. A coating composition comprising
(a) silicon oxide nanoparticles, wherein a perfluorolkylsiloxane is covalently bonded to the silicon oxide nanoparticles;
(b) silver nanoparticles, wherein a perfluorolkylsiloxane is covalently bonded to the silver nanoparticles; and
(c) a solvent.

Aspect 2. The composition of Aspect 1, wherein the silicon oxide nanoparticles have an average particle size of from about 5 nm to about 50 nm.

Aspect 3. The composition of Aspect 1 or 2, wherein the silver nanoparticles have an average particle size of from about 5 nm to about 100 nm.

Aspect 4. The composition of any one of Aspects 1 to 3, wherein the weight ratio of the silicon oxide nanoparticles to the silver nanoparticles is from 0.1 to 5 to 5:0.1.

Aspect 5. The composition of any one of Aspects 1 to 4, wherein the perfluorolkylsiloxane has the formula R²—Si(OR¹)₃, wherein R¹ is a substituted or unsubstituted C₁-C₂₀ alkyl group, and R² is a C₁-C₂₀ perfluoroalkyl group.

Aspect 6. The composition of Aspect 5, wherein R¹ is a C₁ to C₄ alkyl group.

Aspect 7. The composition of Aspect 5, wherein R² is a C₁ to C₁₀ perfluoroalkyl group.

Aspect 8. The composition of Aspect 5, wherein each R¹ is methyl or ethyl, and R² is a C₈ perfluoroalkyl group.

Aspect 9. The composition of any one of Aspects 1 to 3, wherein the silicon oxide nanoparticles and the silver nanoparticles are from 1 weight percent to about 20 weight percent of the composition.

Aspect 10. The composition of any one of Aspects 1 to 9, wherein the organic solvent comprises a hydrocarbon.

Aspect 11. The composition of any one of Aspects 1 to 9, wherein the organic solvent comprises a C₁ to C₁₀ hydrocarbon.

Aspect 12. The composition of any one of Aspects 1 to 9, wherein the composition further comprises an elastomeric polymer.

Aspect 13. The composition of Aspect 12, wherein the elastomeric polymer comprises one or more of a copolyester elastomer, a polyether block amide elastomer, a polyurethane elastomer, a polyolefin based-copolymer elastomer, a styrenic copolymer elastomer, an ionomer elastomer, or any combination thereof.

Aspect 14. The composition of Aspect 12 or 13, the elastomeric polymer is from about 25% to about 75% by weight of the composition.

Aspect 15. The composition of any one of Aspects 1 to 9, wherein the composition further comprises a silicone elastomer and a curing agent.

Aspect 16. The composition of any one of Aspects 1 to 15, wherein the weight ratio of the silicon oxide nanoparticles and the silver nanoparticles to the silicone elastomer and a curing agent is from 0.1 to 5 to 5:0.1.

Aspect 17. A coated article produced by the method comprising
(a) applying the coating composition of any one of Aspects 1 to 16 to at least one surface of the article;
(b) removing the solvent from the coating composition to produce a first coated article; and
(c) applying a nitric oxide releasing compound to the first coated article to produce a second coated article.

Aspect 18. The coated article of Aspect 17, wherein the article is dipped into the coating composition.

Aspect 19. The coated article of Aspect 17, wherein the coating composition is sprayed on at least one surface of the article.

Aspect 20. The coated article of any one of Aspects 17 to 19, wherein the solvent is removed by evaporation.

Aspect 21. The coated article of any one of Aspects 17 to 20, wherein step (b) comprises heating the coated article at a temperature of from about 80° C. to about 120° C. to remove the solvent.

Aspect 22. The coated article of any one of Aspects 17 to 21, wherein the nitric oxide releasing compound is a S-nitrosothiol compound.

Aspect 23. The coated article of any one of Aspects 17 to 21, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

Aspect 24. The coated article of any one of Aspects 17 to 23, wherein the first coated article is dipped into a solution comprising the nitric oxide releasing compound.

Aspect 25. The coated article of any one of Aspects 17 to 23, wherein a composition comprising the nitric oxide releasing compound is sprayed on at least one surface of the first coated article.

Aspect 26. The coated article of any one of Aspects 17 to 25, wherein after step (c), the second coated article is rinsed and dried.

Aspect 27. A coated article comprising a coating on at least one surface of the article, wherein the coating comprises
(a) silicon oxide nanoparticles, wherein a perfluorolkylsiloxane is covalently bonded to the silicon oxide nanoparticles;
(b) silver nanoparticles, wherein a perfluorolkylsiloxane is covalently bonded to the silver nanoparticles; and
(c) a nitric oxide releasing compound.

Aspect 28. The coated article of Aspect 27, wherein the silicon oxide nanoparticles have an average particle size of from about 5 nm to about 50 nm.

Aspect 29. The coated article of Aspect 27, wherein the silver nanoparticles have an average particle size of from about 5 nm to about 100 nm.

Aspect 30. The coated article of any one of Aspects 27 to 29, wherein the weight ratio of the silicon oxide nanoparticles to the silver nanoparticles is from 0.1 to 5 to 5:0.1.

Aspect 31. The coated article of any one of Aspects 27 to 30, wherein the perfluorolkylsiloxane has the formula R²—Si(OR¹)₃, wherein R¹ is a substituted or unsubstituted C₁-C₂₀ alkyl group, and R² is a C₁-C₂₀ perfluoroalkyl group.

Aspect 32. The coated article of Aspect 31, wherein R¹ is a C₁ to C₄ alkyl group.

Aspect 33. The coated article of Aspect 31, wherein R² is a C₁ to C₁₀ perfluoroalkyl group.

Aspect 34. The coated article of Aspect 31, wherein each R¹ is methyl or ethyl, and R² is a C₈ perfluoroalkyl group.

Aspect 35. The coated article of any one of Aspects 27 to 34, wherein the composition further comprises a silicone elastomer.

Aspect 36. The coated article of Aspect 35, wherein the silicone elastomer is from 1 weight percent to about 20 weight percent of the coating.

Aspect 37. The coated article of Aspect 36, wherein the weight ratio of the silicon oxide nanoparticles and the silver nanoparticles to the silicone elastomer is from 0.1 to 5 to 5:0.1.

Aspect 38. The coated article of any one of Aspects 27 to 37, wherein the nitric oxide releasing compound is a S-nitrosothiol compound.

Aspect 39. The coated article of any one of Aspects 27 to 37, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, S-methyl nitrosothioglycolate, nitrosated cysteine, or any combination thereof.

Aspect 40. The coated article of any one of Aspects 27 to 39, wherein the coating further comprises an elastomeric polymer.

Aspect 41. The coated article of Aspect 40, wherein the elastomeric polymer comprises one or more of a copolyester elastomer, a polyether block amide elastomer, a polyurethane elastomer, a polyolefin based-copolymer elastomer, a styrenic copolymer elastomer, an ionomer elastomer, or any combination thereof.

Aspect 42. The coated article of Aspect 40 or 41, the elastomeric polymer is from about 25% to about 75% by weight of the coating.

Aspect 43. The coated article of any one of Aspects 27 to 39, wherein the coating further comprises a silicone elastomer.

Aspect 44. The coated article of any one of Aspects 17 to 43, wherein the article comprises a polymeric grade material, a medical device, a surface or article in a hospital or medical facility, or a surface in an automobile, boat, or aircraft.

Aspect 45. The coated article of any one of Aspects 17 to 43, wherein the article has a water contact angle of from about 75 degrees to about 125 degrees.

Aspect 46. The coated article of any one of Aspects 17 to 43, wherein the article has an advancing contact angle of about 150 degrees to about 250 degrees.

Aspect 47. The coated article of any one of Aspects 17 to 43, wherein the article has a water sliding angle of from about 0.1 degrees to about 5 degrees.

Aspect 48. The coated article of any one of Aspects 17 to 43, wherein the article has a contact angle hysteresis of from about 0.1 degrees to about 5 degrees.

Aspect 49. The coated article of any one of Aspects 17 to 43, wherein the article releases nitric oxide for at least 7 days.

Aspect 50. The coated article of any one of Aspects 17 to 43, wherein the rate of release of nitric oxide from the article is 1 to 5 times less than the same article coated with the coating composition in the absence of silver nanoparticles.

Aspect 51. The coated article of any one of Aspects 17 to 43, wherein the rate of release of nitric oxide from the article is 1 to 5 times less than the same article coated with only the nitric oxide releasing compound.

Aspect 52. The coated article of any one of Aspects 17 to 43, wherein the article is biocompatible.

Aspect 53. The coated article of any one of Aspects 17 to 52, wherein the article prevents the growth of bacteria on at least one surface of the article.

Aspect 54. The coated article of any one of Aspects 17 to 52, wherein the article prevents the adhesion of platelets on at least one surface of the article.

Aspect 55. The coated article of any one of Aspects 17 to 54, wherein the article is an implantable device.

Aspect 56. A method for preventing the growth of bacteria on an article, the method comprising
(a) applying the coating composition of any one of Aspects 1 to 16 to at least one surface of the article and
(b) removing the solvent from the coating composition to produce a first coated article; and
(c) applying a nitric oxide releasing compound to the first coated article to produce a second coated article.

Aspect 57. A method for preventing the adhesion of platelets on an article, the method comprising
(a) applying the coating composition of any one of Aspects 1 to 16 to at least one surface of the article and
(b) removing the solvent from the coating composition to produce a first coated article; and
(c) applying a nitric oxide releasing compound to the first coated article to produce a second coated article.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

Silicon oxide nanoparticles (10-20 nm particle size) were purchased from SkySpring Nanomaterials, Inc. (Houston, USA). 3,3′,5,5′-tetramethylbenzidine (TMB), Phosphate buffered saline (PBS, pH=7.4), Luria Bertani (LB) broth and agar, hexane, tetrahydrofuran, anhydrous ethanol, ethylenediaminetetraacetic acid (EDTA), methanol, silver nanopowder (<100 nm particle size), containing polyvinylpyrrolidone (PVP) as dispersant were obtained from Sigma-Aldrich (St. Louis, MO, USA). 1H,1H,2H,2H-Perfluorooctyltriethoxysilane was purchased from Oakwood Chemical, Inc. (Estill, South Carolina, USA). S-nitroso-N-acetylpenicillamine (SNAP) was obtained from PharmaBlock (Hatfield, PA). Silicone rubber (Sylgard 184) was obtained from Ellsworth Adhesives (USA). Escherichia coli (E. coli ATCC® 25922™) and Staphylococcus aureus (S. aureus ATCC® 6538™) were purchased from American Type Culture Collection (ATCC, Manassas, VA, USA). All chemicals were analytical-grade reagents and utilized without further purification.

### Preparation of Superhydrophobic Particles

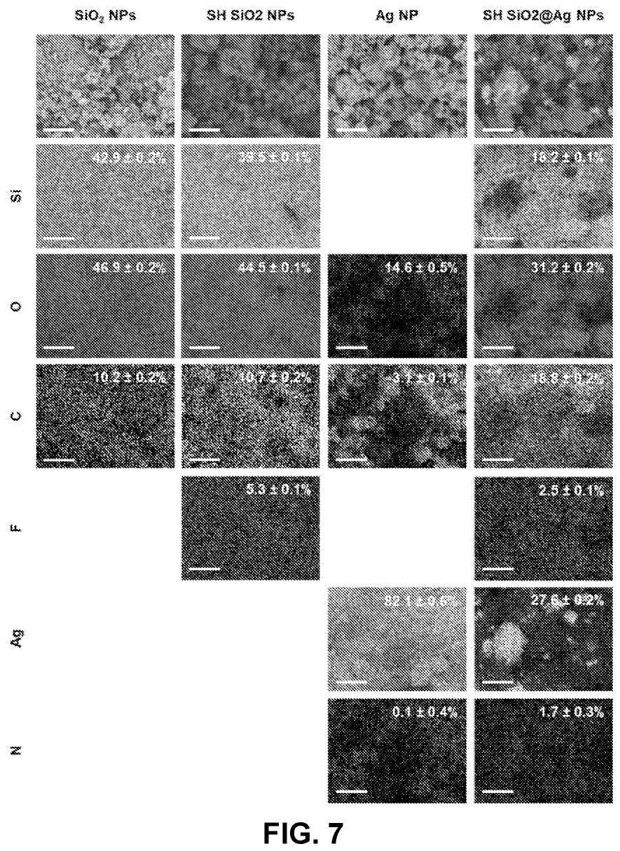

0.5 g of FAS was mixed with 50.0 g of anhydrous ethanol for 10 min with constant agitation at room temperature. Then, SiO₂ (2.0 g) or SiO₂+Ag NPs (2.0 g/1.0 g) were added to the solution, and it was stirred for 10 min followed by sonication for 60 min. After the ethanol was evaporated off, FAS-treated SiO₂ (SH—SiO₂) and SiO₂+Ag (SH—SiO₂+Ag) NPs were obtained and characterized by scanning electron microscopy (SEM), energy-dispersive X-ray spectroscopy (XRD), transmission electron microscopy (TEM), and Fourier-transform infrared spectroscopy, indicating that low-surface-energy FAS molecules were successfully grafted on the surface of SiO₂ and Ag NPs (FIG. 7-9).

### Fabrication of Superhydrophobic, NO-Releasing Substrates

First, 0.75 g of FAS-modified hydrophobic SiO₂ or SiO₂+Ag NPs were ultrasonically dispersed in 10 mL of hexane for 1 h. Afterward, 1.0 g of Sylgard 184 base and 0.1 g of curing agent were added to the paint-like solutions (FIG. 10) and mixed for 15 min. Next, medical-grade silicone rubber substrates were dipped into the above solutions before curing at 100° C. for 3 h. Subsequently, the resulting superhydrophobic substrates were immersed in a SNAP-tetrahydrofuran solution (25 mg/mL) for 2 h (FIG. 10). Finally, the substrates were rinsed with methanol to remove excess SNAP crystals and then air-dried in the dark for 24 h. The fabricated samples have been abbreviated for ease of reference, as demonstrated in Table 1.

TABLE 1
Labeling of various SR samples.

| | | | SNAP |
|---|---|---|---|
| Sample ID | SiO₂ solution | SiO₂ + Ag solution | (NO donor) |
| Silicone | − | − | − |
| rubber (SR) | | | |
| SH-Si | + | − | − |
| SH-SiAg | − | + | − |
| NO | − | − | + |
| SH-SiNO | + | − | + |
| SH-SiAgNO | − | + | + |

### Surface Morphology and Chemistry Analysis

Physical characterizations were carried out using several instrumentation methods. Polymer surface morphology was investigated using Scanning Electron Microscopy (SEM, FEI Teneo, FEI Co.). All samples were coated with gold-palladium (10 nm thickness) using a Leica sputter coater and imaged under an accelerating voltage of 10 kV. The elemental composition of the polymer samples was also studied using an energy-dispersive X-ray spectrometer (EDS, Oxford Instruments) connected to the SEM with an applied voltage of 20.00 kV. The purity and crystalline nature of the samples were studied by X-ray diffraction (XRD) with a Bruker D8 Advance diffractometer equipped with CoKα1 radiation (λ=1.78890 A) operated at 40 kV and 40 mA in the 20 range from 10° to 80° with a scanning rate of 0.1° s⁻¹. Fourier transform infrared spectroscopy was carried out using a Spectrum 3 spectrometer (Perkin-Elmer) using the quantitative potassium bromide (KBr) loading method. Nanoparticle samples were blended with KBr to less than 0.1 wt % and ground down to a uniform consistency. The powder mixture was then cast into 7 mm pellets using a 2-ton press. Samples were analyzed via FTIR from a range of 4000 to 650 cm⁻¹ with a resolution of 4 cm⁻¹. A total of 128 scans were recorded for each sample type.

### Surface Wettability

The anti-wetting properties of the bare and modified SR surfaces were assessed by video capture and processing using a contact angle goniometer and corresponding software from Ossila (Sheffield, UK). The SR segments were cut into square specimens (~1 cm²) and placed on a stage positioned alongside a motion capture camera. Deionized water (5 μL) was then dispensed onto the SR surface with video capture enabling frame-by-frame visualization of the water droplet shape as it came into contact with the surface. Using an edge detection and fitting algorithm, the contact angle of the water droplet was captured in each video frame and used to calculate an average angle for each run. Final data for static contact angle measurements are reported as the mean±standard deviation (SD, n=5 for each sample type).

Sliding angle measurements were similarly performed filming the application of water droplets (5 μL) to the SR samples secured to a stage with a gradually increasing incline. Using video capture software, the exact frame at which the water droplet began to slide off the surface was captured and used to calculate the sliding angle with ImageJ software (NIH). Final data for sliding angle measurements are reported as the mean sliding angle±SD (n=10 for each sample).

Contact angle hysteresis was calculated by measuring the advancing and receding angles of water droplets on the surfaces of the SR samples. Average advancing and receding angles were determined via video capture and analysis of water droplets (5 μL) dispensed and withdrawn from the surface. Hysteresis was then calculated as the difference between the average receding angle and the average advancing angle for each run. Final data are reported as the mean hysteresis measurement±SD (n=5 for each sample type).

### Nitric Oxide Release

Nitric oxide released from the SNAP within the polymer samples was quantified using chemiluminescence detection methods, the gold standard for NO detection. In brief, NO release from the sample types containing SNAP was quantified by immersing the films in PBS buffer (1×, pH=7.4) with EDTA (100 μM) within an amber sample chamber. The amber sample chamber prevented the light reaction of SNAP and was maintained at 37° C. using a water bath to mimic physiological conditions. Any NO released by the samples was purged from the chamber and into the reaction cell by a flow of nitrogen at 200 mL/min. Within the reaction cell, NO reacts with ozone (O₃) to produce an excited state of nitrogen dioxide (NO₂*). When NO₂ drops back down to the ground state, a photon is released that is multiplied and correlated into a voltage and corresponding ppb reading. Utilizing a calculated NOA constant (mol ppb⁻¹ s⁻¹) and the surface area of the sample, the final NO surface flux (×10⁻¹⁰ mol cm⁻² min⁻¹) is calculated. Once readings began at hour 0, all samples were always maintained at 37° C. and in the dark.

### SNAP and Silver Leaching

The loading of SNAP into each sample type was determined by soaking samples (n=5) in THF for 24 h, followed by taking an absorbance reading at 340 nm, the characteristic RSNO peak. The absorbance value was correlated to mg SNAP based on a standard curve of known SNAP concentrations in THF, then normalized to mg per polymer sample. The leaching of SNAP from the polymer films can affect NO release as well as antibacterial effects and cell viability. Therefore, SNAP leaching was measured by soaking SNAP-containing samples in PBS solutions, followed by UV-Vis Spectroscopy of the leachate solutions at 340 nm. A standard curve of SNAP in PBS at known concentrations was used to calculate the SNAP (mg) leached from each film. SNAP loading measurements were then used to determine the percentage of SNAP leached from the samples over 144 h.

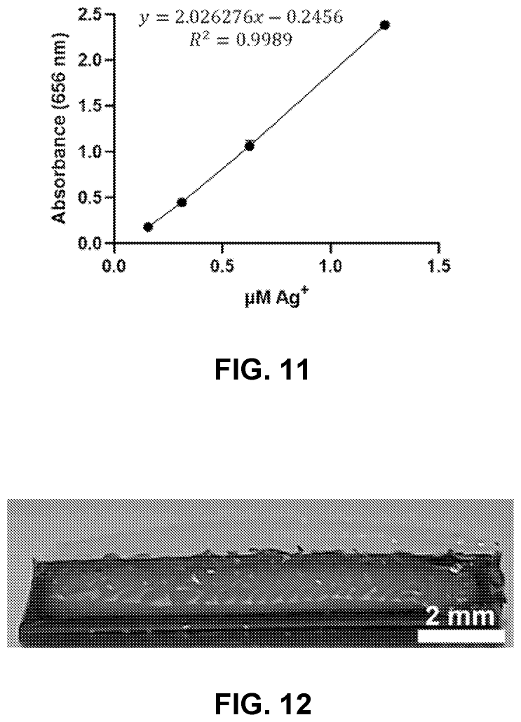

A colorimetric analysis was done to quantify the amount of Ag⁺ present in the leachate solution [53]. A standard solution containing silver nitrate (AgNO₃) was prepared in ultra-pure water. In a typical run, 0.2 mL of AgNO₃ standard solution was mixed with 100 μL of TMB solution (10 mM in ethanol) and 200 μL of sodium acetate buffer solution (pH=4). The samples were incubated at room temperature for 30 minutes, and UV-Visible spectra were recorded at peaks of oxidized TMB (~370 and 655 nm) and visually detected due to the color change from yellow to blue in the presence of Ag+ ions. A standard curve of known Ag+ concentrations was used to quantify released Ag+ (FIG. 11).

### Sterilization Stability

The ability of the proposed material to withstand sterilization conditions was tested. Sterilization was conducted at the University of Georgia College of Veterinary Medicine using a Steris VHP MD140X. In short, SH—SiAgNO samples underwent a 28 min sterilization period via hydrogen peroxide vapor. The sterilizer chamber was filled with hydrogen peroxide that vaporized to a concentration of 6 mg L⁻¹. Once the 28 min sterilization period is complete, all remaining hydrogen peroxide was aerated out of the system. After sterilization and aeration were complete, SH—SiAgNO samples were characterized for SNAP loading and WCA measurement. Differences in values were investigated to determine the sterilization stability of the films.

### Biological Characterizations

### Antibacterial Activity

The dual release of Ag NPs and NO from the fabricated films and the antifouling nature of the superhydrophobic nanoparticle coating affords a multi-pronged strategy to prevent bacterial adhesion and induce bacterial killing if adhesion does occur. To quantify the reduction in bacterial adhesion, a single E. coli bacteria colony was isolated, inoculated into LB media, and grown at 37° C. with orbital shaking (150 rpm) until it reached the log phase of growth. The suspension was centrifuged to yield a bacteria pellet that was rinsed with PBS, and then resuspended in PBS at a concentration of ~10⁸ CFU mL⁻¹. All films (n=3 per sample type) were UV sterilized for 15 minutes on each side. Then, the films were added to a 48-well plate, and 1 mL of the prepared bacterial suspension was added to the wells. The well plate was sealed with parafilm and incubated in the dark at 37° C. and 150 rpm for 24 h. Following 24 h exposure, films were lightly rinsed with PBS and homogenized for 1 min. The homogenate was diluted in PBS and plated onto LB agar plates. After ~18 h incubation of the plates at 37° C., CFUs were counted, and bacterial reduction was quantified (Equation 1) and normalized for the surface area of the samples. The study was also conducted against S. aureus to determine the antimicrobial efficacy against a Gram-positive microorganism.

$$Reduction in Viable Bacteria ( \% ) = \frac{control CFU {mL}^{- 1} - treatment CFU {mL}^{- 1}}{control CFU {mL}^{- 1}} \times 100. \qquad Equation 1$$

### Blood Compatibility-Blood Platelet Adhesion

All protocols about the use of whole blood and platelets were approved by the University of Georgia Institutional Animal Care and Use Committee. Fresh porcine blood was drawn with 3.2% sodium citrate at a ratio of 9:1 (blood:citrate). The anticoagulated blood was centrifuged at 300 rcf for 12 min using a Beckman Coulter Allegra X-30R Centrifuge, and the platelet-rich plasma (PRP) was thereafter collected with a pipette without disturbing the buffy coat layer. To get platelet-poor plasma (PPP), the remaining blood was again centrifuged at 4000 rcf for 20 min. Platelet concentration in both the PRP and PPP fractions was determined using an Element HT5 Veterinary Hematology Analyzer. Based on those concentrations, the PRP and PPP were combined to produce a final platelet concentration of 2×10⁸ platelets mL⁻¹. Samples were briefly placed in phosphate buffer to bring samples to pH equilibrium before platelet exposure. To reverse anticoagulation, calcium chloride was added to the diluted PRP to yield a final concentration of 2.5 mM. 4.0 mL of the calcified platelet solution was then added to a blood tube, which was followed by n=4 of equilibrated samples for each group. Samples were then incubated at 37° C. for 90 min with mild rocking (25 rpm) for 90 mins. Thereafter, samples were gently rinsed with phosphate buffer to remove unbound platelets, placed in Triton-X-phosphate buffer solution (2% v/v Triton-X-100 in phosphate buffer) for 30 mins, and quantified for platelet adhesion via the LDH of the lysed platelets using a Roche Cytotoxicity Detection Kit. A calibration curve was constructed using known dilutions of platelets, and the degree of platelet adhesion on samples was interpolated using this calibration curve.

### Blood Compatibility-Hemolysis

The North American Science Associates (NAMSA) protocol was followed for the evaluation of experimental hemolytic activities. Fresh porcine blood was obtained, and its plasma hemoglobin level was determined using an Element HT5 Veterinary Hematology Analyzer. This whole blood was diluted with Ca²⁺ and Mg²⁺-free (CMF)-PBS to achieve a final hemoglobin concentration of 10 mg/mL. One mL of the diluted whole blood was combined with 7 mL of CMF-PBS for individual sample incubations. This mixture (sans sample) was also used as a negative control. A positive control of diluted whole blood (1 mL) and sterile DI water (7 mL) was also prepared. The treated samples and controls were placed in an incubator at 37° C. for three hours and gently inverted every 30 min. After incubation, the vials were centrifuged at 800×g for 15 min to leave an RBC-free supernatant. The supernatant, which contained any freed hemoglobin from lysed RBCs, was combined with Drabkin's reagent (1:1) and allowed to react for 15 min before reading the absorbance at 540 nm. Percent hemolysis was then calculated using Equation 2.

$$Percent Hemolysis ( \% ) = \frac{Absorbance of sample - Absorbance of blank}{Absorbance of positive control - Absorbance of blank} \times 100 \qquad Equation 2$$

### Cytocompatibility Assessment

The cytocompatibility of all film leachates with mouse fibroblast cells (NIH 3T3 ATCC 1658) was assessed using a colorimetric MTT assay and fluorescence imaging using Calcein-AM and EthD-III stains. MTT reagent (3-(4,5-Dimethyl-2-thiazolyl)-2,5-diphenyl-2H-tetrazolium bromide) forms a water-insoluble blue formazan in the presence of mitochondrial dehydrogenase enzyme. The amount of formazan can be measured spectrophotometrically and is indicative of the metabolically active cells. Mouse fibroblast cells were cultured in tissue culture flasks with Dulbecco's modified Eagle's medium (DMEM) supplemented with FBS (10%) and penicillin-streptomycin (1%) in a humidified incubator at 37° C. with 5% CO₂. The cells were harvested using trypsin upon reaching confluency and collected by centrifuging at 500 rcf for 10 minutes at room temperature. For the cytotoxicity assay, mouse fibroblast cells (5×10³ cells per well) were seeded on tissue culture-treated 96-well plates and incubated overnight at 37° C. with 5% CO₂. Extracts from the samples were prepared by soaking the samples in complete DMEM (1 mL of medium per 10 mg sample) and incubated at 37° C. for 24 h. The next day, the cells were exposed to 10 μL of leachates and further incubated for 24 h. The viability after 24 h of leachate exposure was measured using an MTT assay. The media was removed from wells, and 100 μL of MTT reagent was added to the wells and incubated for 4 h to allow formazan formation. The MTT reagent was then removed from the wells, and the formazan precipitate was dissolved in 100 μL of DMSO. The absorbance was measured at 570 and 690 nm using a multi-plate reader (Biotek Cytation 5). The delta values were calculated as Abs₅₇₀-Abs₆₉₀. The absorbance of treated cells was compared with untreated control cells. The percent viability of the treated cells was calculated using the following formula after subtracting blank absorbance.

$$Cell Viability ( \% ) = \frac{Absorbance of sample}{Absorbance of positive control} \times 100 \qquad Equation 3$$

Fluorescence microscopy was performed using a live dead staining kit (Biotium). Calcein-AM is a cell-permeable dye that produces a green fluorescence after being cleaved by intracellular esterase enzymes. As these enzymes are active only in live cells, Calcein-AM only labels live cells. On the other hand, Ethd-III is a cell impermeable nucleic acid binding stain that labels dead cells. 3T3 cells were seeded on chamber slides (Nunc Lab Tek, Thermo Fisher Scientific, USA) and allowed to form a monolayer. Leachates (1 mg mL⁻¹) were added to the wells and incubated for 24 h at 37° C. with 5% CO₂. After incubation, the adhered cells were rinsed with PBS and stained with Calcein-AM and Ethd-III (5 μM each) for 30 minutes at 37° C. The stained cells were washed with PBS to remove any unbound stain and observed under a confocal laser microscope (Zeiss, LSM 710, USA). Calcein-AM Ex/Em=493 nm/520 nm; EthD-III Ex/Em=553 nm/568 nm.

### Statistical Analysis

All data are reported as mean±standard deviation unless otherwise noted. Statistical comparisons among sample types were performed using ordinary one-way analysis of variance (ANOVA) with corrections for multiple comparisons using Tukey's method, except NOA measurements which were analyzed using two-way ANOVA. Bacterial statistical analysis was performed on the log values of CFU cm⁻² following treatment. P values<0.5 were deemed significant.

### Results and Discussion

### Fabrication of Superhydrophobic, NO-releasing Substrates

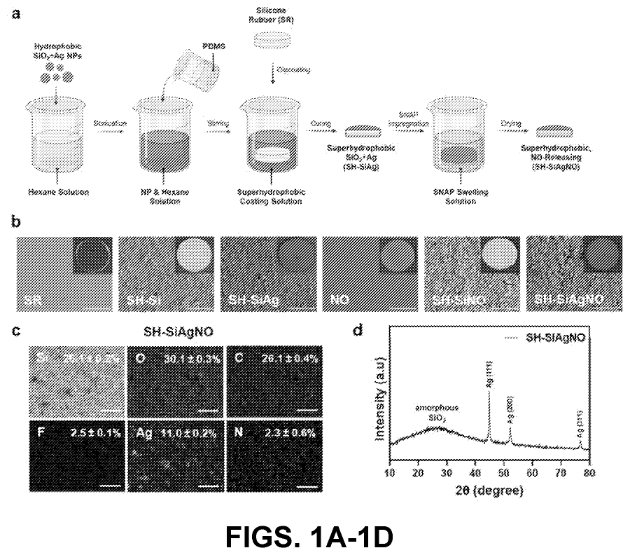

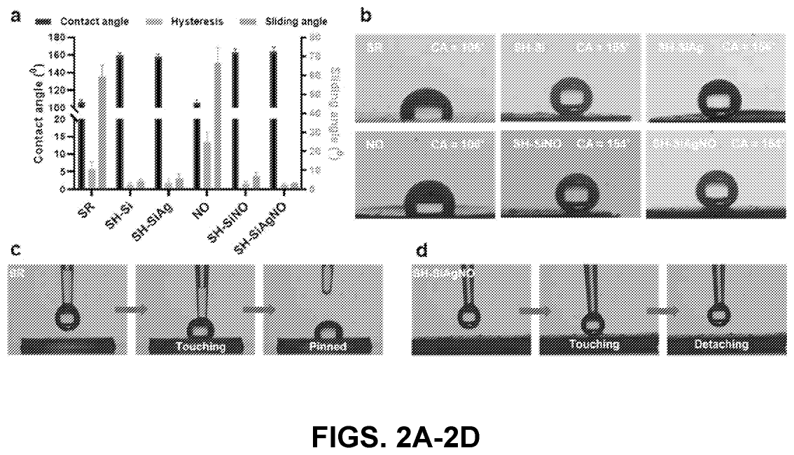

FIG. 1a shows the two-step fabrication process for preparing the superhydrophobic, Ag/NO-releasing silicone polymers for blood-contacting devices. In the first step, a SH—SiAg coating was deposited onto medical-grade SR surfaces by a facile dip-coating method, resulting in a change in the visual appearance of the pristine SR from transparent to dark grey-brown (FIG. 1b). Subsequently, SNAP was incorporated into the coated substrates via solvent impregnation. The resulting SH—SiAgNO was observed to be visually light grey (FIG. 1b). In addition, as visually demonstrated in FIG. 12, this process enabled SNAP incorporation throughout the bulk of the polymer.

### Surface Morphology and Chemistry Analysis

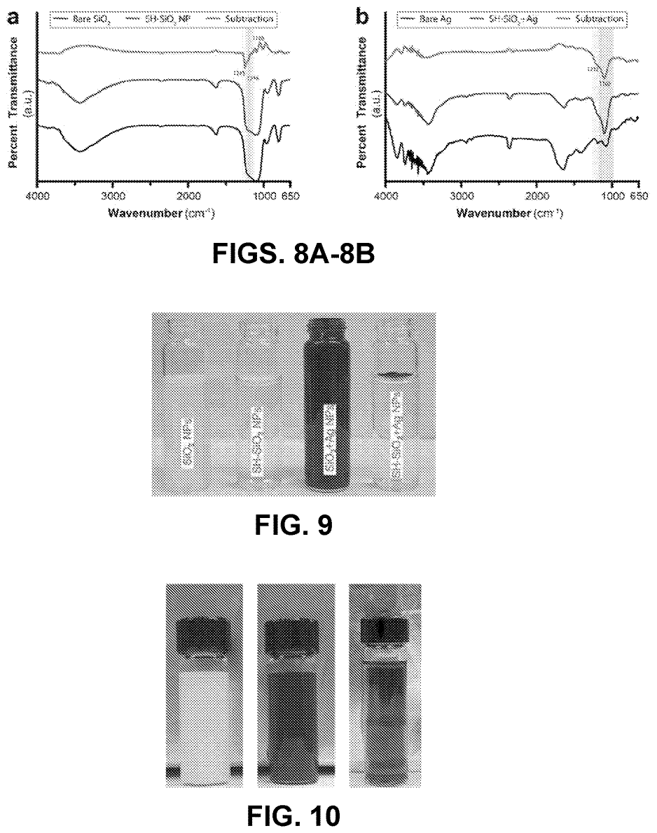

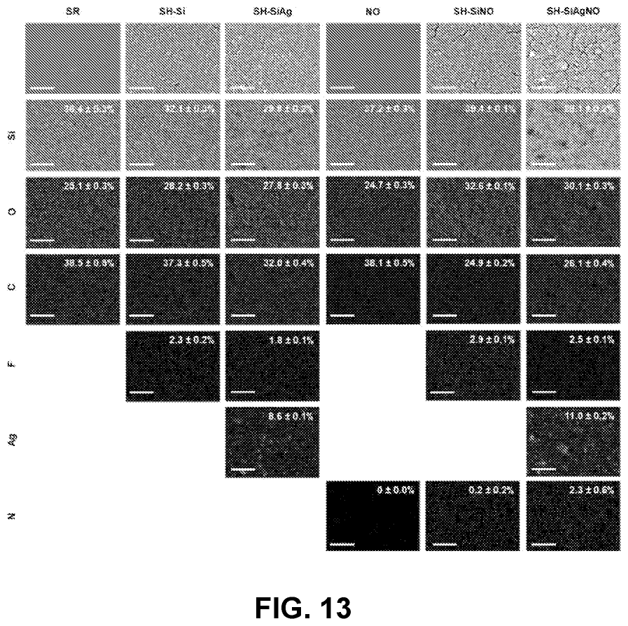

Since surface roughness with low surface energy plays an essential role in entrapping air and conferring superhydrophobicity [54,55], the surface morphology and chemical composition of the fabricated samples were examined using SEM and EDS. As shown in FIG. 1b, the incorporation of SNAP into the SR matrix did not cause any visible change to surface morphology. By contrast, all dip-coated surfaces exhibited hierarchical re-entrant rough structures with voids owing to the coalescence of the dual-sized SiO₂ and Ag NPs. The surface chemical composition of SH—SiAgNO was examined, as shown in FIGS. 1c and 13. Besides the main surface components, including Si, O, and C derived from the SR substrate, F, and Ag elements were detected on SH—SiAgNO, indicating the successful deposition of uniformly distributed FAS-treated SiO₂+Ag NPs on the surface. FTIR analysis of the untreated and FAS-modified nanoparticles further showed the emergence of peaks consistent with surface fluorination, including symmetric C—F and Si—O—C bond stretching with FAS-modified SiO₂ (1245/1216, and 1146 cm⁻¹) and Ag (1212 and 1102 cm⁻¹) nanoparticles, in agreement with prior literature for organic silane-based surface fluorination (FIG. 8) [39,40,56]. The crystal structure of SH—SiAgNO was investigated by XRD (FIG. 1d), showing a broad peak at 2θ=20~30°, which is the characteristic peak of amorphous SiO₂ [57]. In addition, three characteristic peaks of crystalline Ag from SH—SiAgNO were detected at 2θ of 44.8°, 52.1°, and 76.8°, corresponding to (111), (200), and (220) planes, respectively [58].

### Surface Wettability

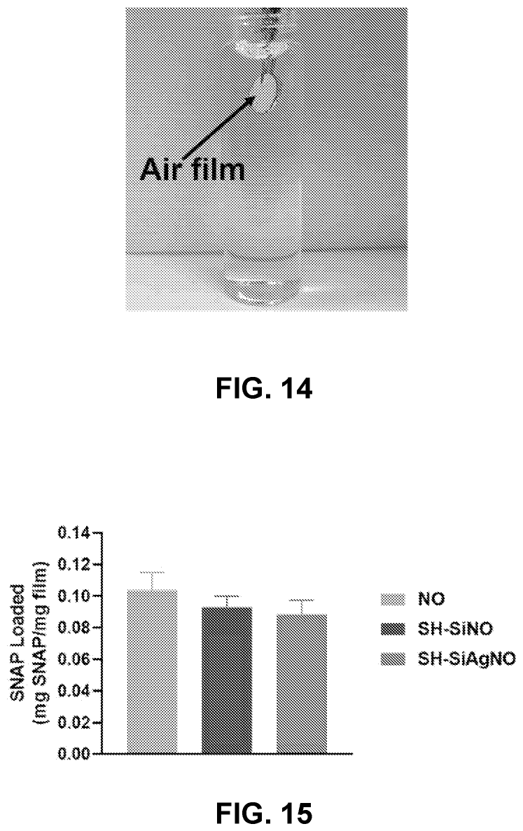

The water contact angles (WCAs), water sliding angles (WSAs), and contact angle hysteresis (CAH) were measured to assess the wettability of all SR surfaces. As demonstrated in FIG. 2a-b, the uncoated SR, which possesses low surface energy, showed a typical hydrophobic character with a WCA of 106.3±2.7° due to the smooth nature of the surface. After incorporating SNAP into the SR matrix, no change in hydrophobicity was observed (105.7±3.1°), which is in good agreement with other reports [49,59]. As expected, after coating with FAS-treated NPs, all surfaces showed strong water-repellency properties, exhibiting WCAs>150° with WSAs) (<5° and CAH) (<5°). This wettability conversion from hydrophobicity to superhydrophobicity is attributed to the combination of the micro/nanoscale morphologies bestowed by the NP clusters and the low surface free energy from FAS. The water droplet strongly adheres to the hydrophobic SR surface, while it can easily move on and detach from the SH—SiAgNO surface owing to the entrapped air in the rough structures, thereby reducing the area of contact (FIG. 14).

### Nitric Oxide Release

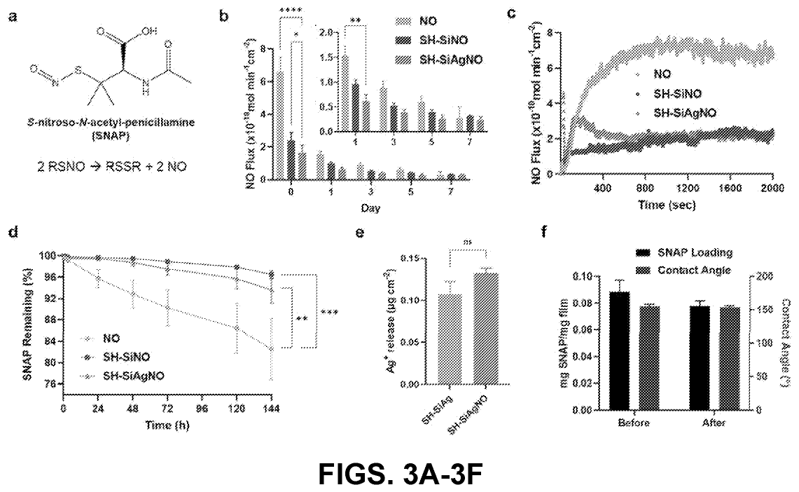

Chemiluminescence-based analytical methods are the gold standard for NO release measurements as they allow for the specific detection of NO radicals. The quantification of NO release from a biomaterial is essential, as NO has shown dose-dependent activity among bacteria and mammalian cells. NO is released from the SNAP molecule through hydrolytic cleavage as well as in the presence of heat, light, and metal ions (FIG. 3a), making it an ideal therapeutic for biomedical applications, as the NO release can be initiated at physiological temperature (37° C.). The NO release from all samples containing SNAP was measured for 7 days (FIG. 3b) under physiological conditions (PBS 10 mM+10 μM EDTA, 37° C.) throughout the study. Over an initial 24 h period, NO samples exhibited a burst release of NO (6.61±0.9×10⁻¹⁰ mol min⁻¹ cm⁻²) compared to SH—SiNO and SH—SiAgNO samples (2.40±0.5×10⁻¹⁰ mol min⁻¹ cm⁻² and 1.64±0.4 10⁻¹⁰ mol min⁻¹ cm⁻², respectively) (FIG. 3c). The difference in NO release profiles can be attributed to the presence of a SH layer that limits the hydration of the underlying SR substrate, which prevents the immediate spike in release commonly associated with NO-releasing materials [60]. NO release slowly decreased over the 7 day period for all samples, as the SNAP reservoir within the samples was slowly depleting. However, on day 7, all films were still releasing NO at concentrations shown to elicit a biological response [61]. SNAP films showed a flux of 0.273±0.23 (×10⁻¹⁰ mol min⁻¹ cm⁻²), SH—SiNO released 0.322±0.01 (×10⁻¹⁰ mol min⁻¹ cm⁻²), and SH—SiAgNO released 0.241±0.06 (×10⁻¹⁰ mol min⁻¹ cm⁻²) on day 7 (p>0.9 for all comparisons). The continuous NO flux at endothelial levels (0.2-4×10⁻¹⁰ mol min⁻¹ cm⁻²) from the treated polymers for 7 days demonstrates their potential as effective antimicrobial and hemocompatible materials for a variety of medical devices.

### SNAP and Silver Leaching

The versatility of the SNAP molecule allows for facile impregnation into a polymer film in the proper solvent and polymer system. NO release from SNAP imbues bioactivity to otherwise inert materials. However, the leaching of SNAP from polymer films must be quantified and controlled, as minute leaching allows for antibacterial effects in the surrounding areas, but excessive leaching can elicit a cytotoxic response in mammalian cells. Therefore, the leaching of SNAP from the prepared materials was studied by first quantifying the SNAP loading into each of the sample types (FIG. 15). There was no significant difference in the amount of SNAP loaded into the samples, indicating that the presence of SH coatings had no significant negative effects on the swelling behavior of the polymer samples. Second, SNAP leaching was quantified from all NO-releasing films for 7 d. Overall, NO films showed the greatest SNAP leaching with only 82.53±5.72% remaining after 7 days in physiological conditions (FIG. 3d). Both SH—SiNO and SH—SiAgNO polymers showed reduced leaching with 96.43±0.70% and 93.64±2.55% SNAP remaining after 7 d. The decreased SNAP leaching from SH—SiNO and SH—SiAgNO films is due to the decrease in water uptake of the films, leading to less water permeation into the polymer matrix and allowing fewer SNAP molecules to leach into the solution. Compared to NO films alone, both SH—SiNO and SH—SiAgNO show more desirable leaching characteristics due to their superhydrophobic surfaces, retaining SNAP molecules within the polymer matrix.

The Ag NPs present in the current fabrication design not only play a role in providing a superhydrophobic surface to minimalize fouling, but their leaching also contributes to the antimicrobial potential of the material. Silver leachates were quantified using UV-Vis spectroscopy and found to be not significantly different between sample types. SH—SiAg samples released 0.107±0.02 μg Ag cm⁻², while SH—SiAgNO samples released 0.132±0.01 μg Ag cm⁻² (FIG. 3e)

### Sterilization Stability

The development of novel materials for biomedical applications is essential for the forward progress of medicine, but there are practical design constraints that cannot be overlooked. The sterilization stability of a biomaterial is vital, as improper sterilization of implanted and external materials leads to direct patient exposure to infectious pathogens. In this study, SH—SiAgNO samples were subjected to hydrogen peroxide (H₂O₂) sterilization and investigated for their ability to maintain SNAP loading and superhydrophobicity. Hydrogen peroxide sterilization is widely used in clinical settings as it sterilizes quickly and effectively without producing toxic residual substances [63]. Before undergoing sterilization, SH—SiAgNO samples displayed an average SNAP loading of 0.088±0.01 mg SNAP per mg film and a superhydrophobic contact angle of 155.38±2.9° (FIG. 3f). Following sterilization, SH—SiAgNO films displayed insignificant changes in SNAP loading (0.078±0.004 mg SNAP per mg film, p>0.9), and the contact angle measured 153.72±2.5° (ns, p>0.3). The ability of the films to maintain SNAP loading shows that the bioactivity of the films will not be lost during or after H₂O₂ sterilization. Further, the maintenance of superhydrophobicity preserves the antifouling nature of the SH—SiAgNO films throughout the sterilization process, displaying the potential for the use of these polymers in clinical healthcare settings.

### Biological Characterizations

### Antibacterial Activity

The antibacterial nature of NO lies in its rapid reactivity with environmental O₂, leading to the production of a wide range of reactive oxygen species (ROS) and reactive nitrogen species (RNS). These molecules induce nitrosative and oxidative stress on microbial systems, leading to membrane rupture, protein structure modification, and disruption of DNA and DNA repair systems [64,65]. Additionally, Ag has been shown to induce structural damage to bacterial membranes as well as deactivate several important membrane enzymes, leading to bacterial death [66]. Ag nanoparticles further inhibit bacterial growth by altering the expression of essential proteins and damaging DNA structures [67]. Both NO and Ag nanoparticles alone elicit potent antimicrobial effects, and the combination has shown enhanced bacterial killing when combined in alginate nanoparticles as well as PVA-PEG films [69]. However, neither NO nor Ag prevents the initial attachment of bacteria to a polymer surface. Therefore, the superhydrophobic surface modification employed here provides a passive antibacterial approach to the two active approaches also included in this novel polymer film. The antifouling nature of superhydrophobic surfaces derives from the low surface energy imparted by the air layer entrapped in the rough surface structures.

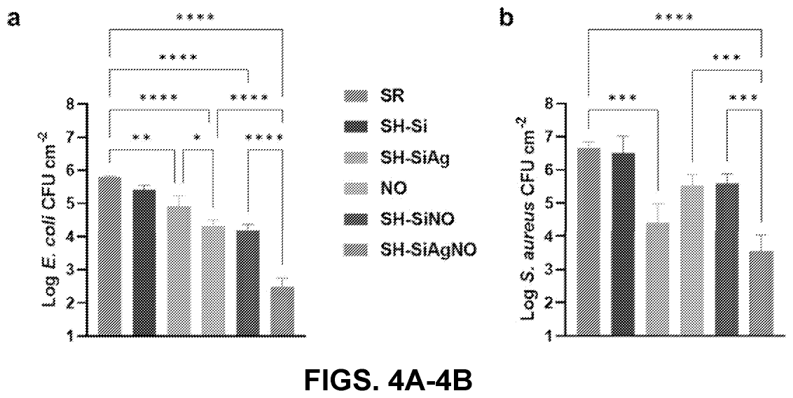

To examine the antibacterial capabilities of SH—SiAgNO films, polymers at every step of the modification process were exposed to E. coli and S. aureus, common Gram-negative and Gram-positive pathogens often associated with HAls. Following 24 h of exposure to E. coli, SH—SiAgNO films showed the greatest reduction in bacterial viability compared to control SR at 99.95±0.03% due to the combination of the anti-adhesive property of the superhydrophobic coating and the bactericidal nature of the released Ag and NO species (FIG. 4a). SH—Si reduced bacterial adhesion, though not significantly (56.53±12.1%), while SH—SiAg, NO, and SH—SiNO displayed an 83.70±11%, 96.43±1.7%, and 97.38±1.0% reduction, respectively (Table 2).

TABLE 2
E. coli bacterial reductions.

| | | | p-value |
|---|---|---|---|
| | | Bacterial | (compared to |
| Sample | CFU cm⁻² | Reduction (%) | control SR) |
| SR | 6.18E+05 ± 5.94E+04 | — | — |
| SH-Si | 2.69E+05 ± 7.48E+04 | 56.53 ± 12.1% | p < 0.307 |
| SH-SiAg | 1.01E+05 ± 7.28E+04 | 83.70 ± 11.8% | p < 0.0024 |
| NO | 2.20E+04 ± 1.04E+04 | 96.43 ± 1.69% | p < 0.0001 |
| SH-SiNO | 1.62E+04 ± 6.54E+03 | 97.38 ± 1.06% | p < 0.0001 |
| SH-SiAgNO | 3.31E+02 ± 1.78E+02 | 99.95 ± 0.03% | p < 0.0001 |

Exposure of all film types to S. aureus led to a similar trend, with SH—SiAgNO films exhibiting the greatest microbial killing with a 99.89±0.13% reduction in bacterial viability (FIG. 4b). The SH—Si surface showed a negligible reduction, while SH—SiAg films reduced bacterial viability by 99.08±1.2%. Both NO and SH—SiNO films showed ~91% bacterial viability reduction (Table 3). Although the final treatment sample (SH—SiAgNO) showed the most potential as an antimicrobial surface, there was a visible decrease in efficacy for the superhydrophobic surfaces against S. aureus colonization. This phenomenon has been shown in previous literature and is likely due to the physical properties of the bacteria itself. Rod-shaped E. coli require a secure attachment over a relatively large surface area to adhere, while spherically shaped S. aureus require less favorable attachment over a smaller surface area to adhere [70,71]. However, the decreased effectiveness of the superhydrophobic surfaces against the Gram-positive bacteria was overcome by the antimicrobial potency of NO gas combined with Ag NP release, as SH—SiAgNO showed a 2-log reduction in bacterial viability compared to controls. The combination of active antibacterial strategies with the passive antifouling nature of the nanoparticle surface coating provides a potent antimicrobial medical device surface, as the low surface energy prevents bacterial adhesion, while the Ag and NO components induce killing to those bacteria that can adhere.

TABLE 3
S. aureus bacterial reductions.

| | | | p-value |
|---|---|---|---|
| | | Bacterial | (compared to |
| Sample | CFU cm⁻² | Reduction (%) | control SR) |
| SR | 4.90E+06 ± 1.9E+06 | — | — |
| SH-Si | 4.55E+06 ± 3.2E+06 | 7.050 ± 66.2% | p > 0.1 |
| SH-SiAg | 4.53E+04 ± 5.6E+04 | 99.08 ± 1.15% | p < 0.001 |
| NO | 3.94E+05 ± 2.4E+05 | 91.96 ± 4.91% | p > 0.05 |
| SH-SiNO | 4.47E+05 ± 2.7E+05 | 90.87 ± 5.45% | p > 0.05 |
| SH-SiAgNO | 5.50E+03 ± 6.2E+03 | 99.89 ± 0.13% | p < 0.0001 |

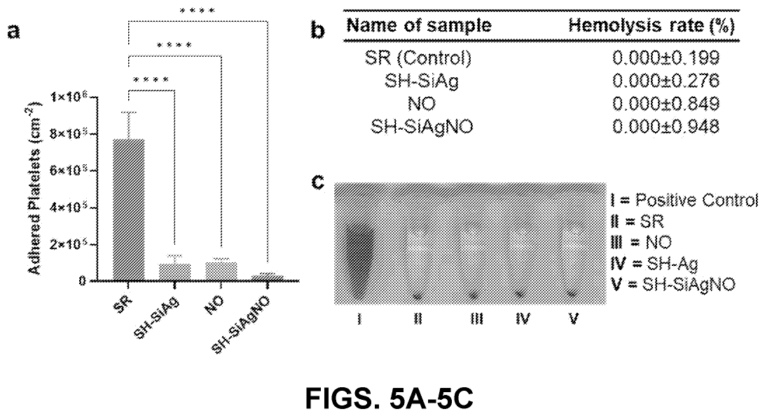

Meanwhile, the hemocompatibility of these surfaces was further evaluated with porcine blood using the ISO 10993-4 protocol [73]. As all samples showed a percent hemolysis of less than 2% by ISO standards, all samples are hence considered nonhemolytic within the 3 h study (FIG. 5b-c). Taken together, these results confirm that the SH—SiAgNO surface possesses good hemocompatibility.

### Cytocompatibility Assessment

Any biomaterial needs to be cytocompatible and not induce any adverse effects on normal mammalian cells, as it would compromise the clinical practicality of the material. These adverse effects may be in the form of cytotoxicity or a change in cell morphology. Specifically for this material, cytocompatibility must be investigated because cytotoxic effects of higher concentrations of silver nanoparticles have been reported [74]. We demonstrated the safety and cytocompatibility of SH—SiAgNO surfaces on mouse fibroblast cells using a colorimetric MTT assay and fluorescence imaging using Calein-AM and EthD-III stains. The cytocompatibility assessment and analysis were performed in accordance with the ISO standards, which define any biomaterial with more than 70% cellular viability as cytocompatible [75]. Leachates extracted from the samples, as recommended for analysis of an indwelling medical device by ISO, were exposed to 3T3 cells for 24 h followed by viability measurement. MTT assay provides the relative viability of cells treated with sample leachates compared to untreated cells and the fluorescence imaging differentially visualized live and dead cells.

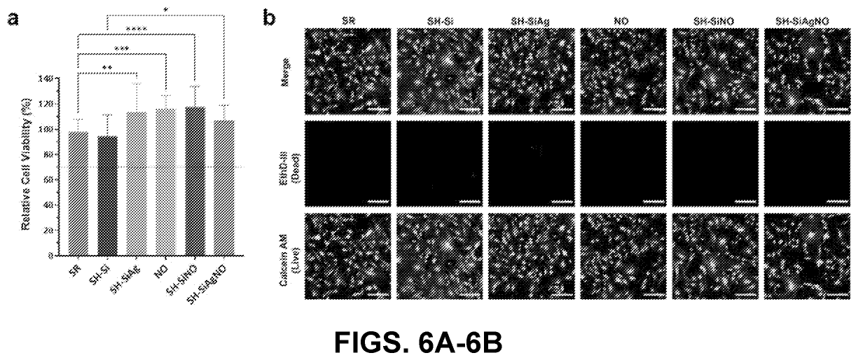

The results demonstrated good cytocompatibility and low toxicity, with all sample types showing more than 70% viability on par with ISO standards (FIG. 6a). Previous studies have found the IC₅₀ value of Ag NPs in primary mouse fibroblast cells to be 61 μg/mL and showed them to be cytocompatible up to 25 μg/mL concentration [76]. As indicated by the Ag⁺ release assay, the SH—SiAg and SH—SiAgNO films had less than 2 μg cm⁻² of Ag⁺ ions released over 24 h, and these concentrations did not exert any toxicity on mouse fibroblast cells. The fluorescence imaging corroborated the MTT data as most of the cells were alive, as shown by Calcein staining. Moreover, a minimal number of cells took up the EthD-III stain indicating a very low number of dead cells following treatment (FIG. 6b). Overall, the surfaces fabricated were cytocompatible with mouse fibroblasts and did not exert any evident toxic effects.

### Blood Compatibility

Blood compatibility is a prerequisite for blood-contacting devices and materials. The adhesion and activation of platelets on medical device surfaces play a major role in thrombus formation [10,72]. Hence, to assess the anti-platelet property of the prepared materials, they were exposed to porcine plasma for 90 min under physiological conditions. As demonstrated in FIG. 5a, the adhesion of platelets was significantly reduced by 87.9±5.8% and 86.9±2.9% on the surfaces of SH—SiAg and NO, respectively, relative to uncoated SR surfaces. The effect of the SH—SiAgNO combination was even more remarkable, reducing platelet attachment by 96.2±1.6% compared to control surfaces. This phenomenon may be attributed to the reduced interaction between platelets and the SH—SiAgNO surface induced by the superhydrophobicity and NO release. Compared to other previously developed NO-releasing biomaterials, the SH—SiAgNO fabricated in this work showed comparable or superior performance in reducing the adhesion of bacteria and platelets (Table 4).

TABLE 4
Comparison of the antimicrobial and antifouling efficiencies
of the SH-SiAgNO samples with other NO-releasing materials.

| | | Reduction in | | |
|---|---|---|---|---|
| | Reduction in adherent | adherent | | |
| Material | bacteria (%) | platelets (%) | Wettability | Ref. |
| LINORel | 99.9% - S. aureus | — | slippery | [1] |
| SR-SNAP-Slip | 99.8% - S. aureus | — | slippery | [2] |
| NO/SH | 99.9% - P. aeruginosa | — | superhydrophobic | [3] |
| Cu GSNO | 99.9% - S. aureus | — | hydrophobic | [4] |
| | 96.8% - P. aeruginosa | | | |
| AMP@Cu-DOTa | 96.4% - S. epidermidis | 96.0% | — | [5] |
| | 96.0% - E. coli | | | |
| Hep-NO-SR | 99.5% - S. aureus | 84.1% | hydrophilic | [6] |
| SNAP-AmB | 99.0% - S. aureus | 74.6% | hydrophilic | [7] |
| | 89.7% - E. coli | | | |
| SNAP-Se | 99.7% - S. aureus | 85.5% | hydrophilic | [8] |
| | 99.2% - E. coli | | | |
| SNAP-PTFE | 99.3% - S. aureus | 83.3% | hydrophobic | [9] |
| | 99.1% - E. coli | | | |
| SH-SiAgNO | 99.0% - S. aureus | 96.2% | superhydrophobic | Current |
| | 99.9% - E. coli | | | study |

LINORel: Liquid-infused nitric oxide-releasing; SR: silicone; SNAP: S-nitroso-N-acetylpenicillamine; Slip: silicone oil; NO: nitric oxide; SH: superhydrophobic; Cu: copper; GSNO: S-nitrosoglutathione; Hep: Heparin; AmB: Amphotericin B; Se: Selenium; PTFE: polytetrafluoroethylene.

### CONCLUSION

In summary, an antimicrobial and hemocompatible SR-based material has been fabricated. The fabrication process involved dip-coating the base polymer with FAS-treated NPs to create a superhydrophobic surface and then solvent swelling SNAP into the materials to incorporate a NO donor molecule. Successful fabrication of the surface coating was revealed by SEM, EDS, and XRD. Nitric oxide release from the SH—SiAgNO films was maintained within physiological levels for 7 days with minimal SNAP leaching, revealing the potential for biological testing. Following hydrogen peroxide sterilization of the samples, SH—SiAgNO films maintained their superhydrophobicity and NO payload, as examined by water contact angle and SNAP loading within the films. Biological testing of the samples showed that the combination of the active NO release and Ag leaching, along with the passive antifouling nature of the superhydrophobic surface, led to the enhanced killing of E. coli and S. aureus compared to SR controls in a 24 h exposure study. Further, adhered blood platelet counts were significantly reduced without exhibiting any signs of lysis of blood cells, confirming hemocompatibility. Lastly, all sample types showed insignificant decreases in mammalian cell viability following 24 h leachate exposure. Overall, the proposed material provides a promising strategy to prevent infection and thrombosis on medical device surfaces by utilizing the antimicrobial potency of NO and Ag NPs combined with the antifouling nature of superhydrophobic surfaces.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

[1] R. O. Darouiche, Device-associated infections: A macroproblem that starts with microadherence, Clin. Infect. Dis. 33 (2001). https://doi.org/10.1086/323130.
[2] P. M. McCarthy, W. A. Smith, Mechanical circulatory support-A long and winding road, Science (80-.). 295 (2002). https://doi.org/10.1126/science.1068555.
[3] J. I. Shen, A. A. Mitani, T. I. Chang, W. C. Winkelmayer, Use and safety of heparin-free maintenance hemodialysis in the USA, Nephrol. Dial. Transplant. 28 (2013). https://doi.org/10.1093/ndt/gft067.
[4] T. Marnejon, D. Angelo, A. A. Abdou, D. Gemmel, Risk factors for upper extremity venous thrombosis associated with peripherally inserted central venous catheters, J. Vasc. Access. 13 (2012). https://doi.org/10.5301/jva.5000039.
[5] L. A. Mermel, M. Allon, E. Bouza, D. E. Craven, P. Flynn, N. P. O'Grady, I. I. Raad, B. J. A. Rijnders, R. J. Sherertz, D. K. Warren, Clinical practice guidelines for the diagnosis and management of intravascular catheter-related infection: 2009 update by the infectious diseases society of America, Clin. Infect. Dis. 49 (2009). https://doi.org/10.1086/599376.
[6] D. Maki, P. A. Tambyah, Engineering out the Risk of Infection with Urinary Catheters, Emerg. Infect. Dis. 7 (2001) 342-347. https://doi.org/10.3201/eid0702.010240.
[7] R. Donlan, Biofilms and Device-Associated Infections, Emerg. Infect. Dis. 7 (2001) 277-281. https://doi.org/10.3201/eid0702.010226.
[8] HAI Data|CDC, (n.d.). https://www.cdc.gov/hai/data/index.html (accessed Dec. 5, 2022).
[9] T. Hilal, J. Mudd, T. G. DeLoughery, Hemostatic complications associated with ventricular assist devices, Res. Pract. Thromb. Haemost. 3 (2019). https://doi.org/10.1002/rth2.12226.
[10] I. H. Jaffer, J. C. Fredenburgh, J. Hirsh, J. I. Weitz, Medical device-induced thrombosis: What causes it and how can we prevent it?, J. Thromb. Haemost. 13 (2015) S72-S81. https://doi.org/10.1111/jth.12961.
[11] R. Gbyli, A. Mercaldi, H. Sundaram, K. A. Amoako, Achieving Totally Local Anticoagulation on Blood Contacting Devices, Adv. Mater. Interfaces. 5 (2018). https://doi.org/10.1002/admi.201700954.
[12] M. B. Gorbet, M. V. Sefton, Biomaterial-associated thrombosis: roles of coagulation factors, complement, platelets and leukocytes, Biomaterials. 25 (2004) 5681-5703. https://doi.org/10.1016/j.biomaterials.2004.01.023.
[13] R. Biran, D. Pond, Heparin coatings for improving blood compatibility of medical devices, Adv. Drug Deliv. Rev. 112 (2017). https://doi.org/10.1016/j.addr.2016.12.002.
[14] I. H. Jaffer, J. I. Weitz, The blood compatibility challenge. Part 1: Blood-contacting medical devices: The scope of the problem, Acta Biomater. 94 (2019). https://doi.org/10.1016/j.actbio.2019.06.021.
[15] E. Ostuni, R. G. Chapman, R. E. Holmlin, S. Takayama, G. M. Whitesides, A Survey of Structure-Property Relationships of Surfaces that Resist the Adsorption of Protein, Langmuir. 17 (2001) 5605-5620. https://doi.org/10.1021/la010384m.
[16] Y. Dai, S. Dai, X. Xie, J. Ning, Immobilizing argatroban and mPEG-NH2 on a polyethersulfone membrane surface to prepare an effective nonthrombogenic biointerface, Https://Doi.org/10.1080/09205063.2019.1595891. 30 (2019) 608-628. https://doi.org/10.1080/09205063.2019.1595891.
[17] S. Zhang, X. Liang, G. M. Gadd, Q. Zhao, A sol-gel based silver nanoparticle/polytetrafluorethylene (AgNP/PTFE) coating with enhanced antibacterial and anti-corrosive properties, Appl. Surf. Sci. 535 (2021) 147675. https://doi.org/10.1016/J.APSUSC.2020.147675.
[18] D. Czerwińska-Główka, W. Przystaś, E. Zabłocka-Godlewska, S. Student, B. Cwalina, M. Łapkowski, K. Krukiewicz, Electrically-responsive antimicrobial coatings based on a tetracycline-loaded poly(3,4-ethylenedioxythiophene) matrix, Mater. Sci. Eng. C. 123 (2021). https://doi.org/10.1016/j.msec.2021.112017.
[19] M. Mazraeh, F. Eshrati Yeganeh, M. Yousefi, S. Baniyaghoob, B. Farasati Far, I. Akbarzadeh, A. Bigham, M. Ashrafizadeh, N. Rabiee, P. Makvandi, M. R. Saeb, Multifunctional Tetracycline-Loaded Silica-Coated Core-Shell Magnetic Nanoparticles: Antibacterial, Antibiofilm, and Cytotoxic Activities, ACS Appl. Bio Mater. (2022) acsabm.2c00100. https://doi.org/10.1021/acsabm.2c00100.
[20] R. G. Shahi, M. T. P. Albuquerque, E. A. Munchow, S. B. Blanchard, R. L. Gregory, M. C. Bottino, Novel bioactive tetracycline-containing electrospun polymer fibers as a potential antibacterial dental implant coating, Odontology. 105 (2017). https://doi.org/10.1007/s10266-016-0268-z.
[21] B. Xie, R. Zhang, H. Zhang, A. Xu, Y. Deng, Y. Lv, F. Deng, S. Wei, Decoration of heparin and bovine serum albumin on polysulfone membrane assisted via polydopamine strategy for hemodialysis, J. Biomater. Sci. Polym. Ed. 27 (2016). https://doi.org/10.1080/09205063.2016.1169479.
[22] C. J. Pan, L. Q. Pang, F. Gao, Y. N. Wang, T. Liu, W. Ye, Y. H. Hou, Anticoagulation and endothelial cell behaviors of heparin-loaded graphene oxide coating on titanium surface, Mater. Sci. Eng. C. 63 (2016). https://doi.org/10.1016/j.msec.2016.03.001.
[23] H. Wei, L. Han, J. Ren, L. Jia, Anticoagulant surface coating using composite polysaccharides with embedded heparin-releasing mesoporous silica, ACS Appl. Mater. Interfaces. 5 (2013). https://doi.org/10.1021/am403882x.
[24] Y. E. Hwang, S. Im, H. Kim, J. H. Sohn, B. K. Cho, J. H. Cho, B. H. Sung, S. C. Kim, Adhesive antimicrobial peptides containing 3,4-dihydroxy-I-phenylalanine residues for direct one-step surface coating, Int. J. Mol. Sci. 22 (2021). https://doi.org/10.3390/ijms222111915.
[25] M. Zabara, Q. Ren, H. Amenitsch, S. Salentinig, Bioinspired Antimicrobial Coatings from Peptide-Functionalized Liquid Crystalline Nanostructures, ACS Appl. Bio Mater. 4 (2021). https://doi.org/10.1021/acsabm. 1c00415.
[26] L. Townsend, R. L. Williams, O. Anuforom, M. R. Berwick, F. Halstead, E. Hughes, A. Stamboulis, B. Oppenheim, J. Gough, L. Grover, R. A. H. Scott, M. Webber, A. F. A. Peacock, A. Belli, A. Logan, F. De Cogan, Antimicrobial peptide coatings for hydroxyapatite: Electrostatic and covalent attachment of antimicrobial peptides to surfaces, J. R. Soc. Interface. 14 (2017). https://doi.org/10.1098/rsif.2016.0657.
[27] Q. Miao, J. L. Sun, F. Huang, J. Wang, P. Wang, Y. F. Zheng, F. Wang, C. F. Ma, Antibacterial Peptide HHC-36 Sustained-Release Coating Promotes Antibacterial Property of Percutaneous Implant, Front. Bioeng. Biotechnol. 9 (2021). https://doi.org/10.3389/fbioe.2021.735889.
[28] L. Hui, J. Huang, G. Chen, Y. Zhu, L. Yang, Antibacterial Property of Graphene Quantum Dots (Both Source Material and Bacterial Shape Matter), ACS Appl. Mater. Interfaces. 8 (2016). https://doi.org/10.1021/acsami.5b10132.
[29] E. G. A. Owusu, E. Yaghini, I. Naasani, I. P. Parkin, E. Allan, A. J. MacRobert, Synergistic interactions of cadmium-free quantum dots embedded in a photosensitised polymer surface: Efficient killing of multidrug-resistant strains at low ambient light levels, Nanoscale. 12 (2020). https://doi.org/10.1039/c9nr10421f.
[30] M. D. Rojas-Andrade, T. A. Nguyen, W. P. Mistler, J. Armas, J. E. Lu, G. Roseman, W. R. Hollingsworth, F. Nichols, G. L. Millhauser, A. Ayzner, C. Saltikov, S. Chen, Antimicrobial activity of graphene oxide quantum dots: impacts of chemical reduction, Nanoscale Adv. 2 (2020). https://doi.org/10.1039/c9na00698b.
[31] R. E. Cronin, R. F. Reilly, Unfractionated Heparin for Hemodialysis: Still the Best Option, Semin. Dial. 23 (2010). https://doi.org/10.1111/j.1525-139X.2010.00770.x.
[32] W. Orapiriyakul, P. S. Young, L. Damiati, P. M. Tsimbouri, Antibacterial surface modification of titanium implants in orthopaedics, J. Tissue Eng. 9 (2018). https://doi.org/10.1177/2041731418789838.
[33] H. Qiu, Z. Si, Y. Luo, P. Feng, X. Wu, W. Hou, Y. Zhu, M. B. Chan-Park, L. Xu, D. Huang, The Mechanisms and the Applications of Antibacterial Polymers in Surface Modification on Medical Devices, Front. Bioeng. Biotechnol. 8 (2020). https://doi.org/10.3389/fbioe.2020.00910.
[34] E. Ozkan, C. C. Crick, A. Taylor, E. Allan, I. P. Parkin, Copper-based water repellent and antibacterial coatings by aerosol assisted chemical vapour deposition, Chem. Sci. 7 (2016) 5126-5131. https://doi.org/10.1039/c6sc01150k.
[35] A. Patir, G. B. Hwang, C. Lourenco, S. P. Nair, C. J. Carmalt, I. P. Parkin, Crystal Violet-Impregnated Slippery Surface to Prevent Bacterial Contamination of Surfaces, ACS Appl. Mater. Interfaces. (2021) acsami.0c17915. https://doi.org/10.1021/acsami.0c17915.
[36] N. Celik, F. Sahin, M. Ruzi, M. Yay, E. Unal, M. S. Onses, Blood repellent superhydrophobic surfaces constructed from nanoparticle-free and biocompatible materials, Colloids Surfaces B Biointerfaces. 205 (2021). https://doi.org/10.1016/j.colsurfb.2021.111864.
[37] J. Zhang, G. Li, D. Li, X. Zhang, Q. Li, Z. Liu, Y. Fang, S. Zhang, J. Man, In Vivo Blood-Repellent Performance of a Controllable Facile-Generated Superhydrophobic Surface, ACS Appl. Mater. Interfaces. 13 (2021). https://doi.org/10.1021/acsami.0c21058.
[38] Z. Li, B. L. Nguyen, Y. C. Cheng, J. Xue, G. MacLaren, C. H. Yap, Durable, flexible, superhydrophobic and blood-repelling surfaces for use in medical blood pumps, J. Mater. Chem. B. 6 (2018). https://doi.org/10.1039/C8TB01547C.
[39] E. Ozkan, A. Mondal, P. Singha, M. Douglass, S. P. Hopkins, R. Devine, M. Garren, J. Manuel, J. Warnock, H. Handa, Fabrication of Bacteria- And Blood-Repellent Superhydrophobic Polyurethane Sponge Materials, ACS Appl. Mater. Interfaces. 12 (2020) 51160-51173. https://doi.org/10.1021/acsami.0c13098.
[40] E. Ozkan, A. Mondal, M. Douglass, S. P. Hopkins, M. Garren, R. Devine, R. Pandey, J. Manuel, P. Singha, J. Warnock, H. Handa, Bioinspired ultra-low fouling coatings on medical devices to prevent device-associated infections and thrombosis, J. Colloid Interface Sci. 608 (2022) 1015-1024. https://doi.org/10.1016/j.jcis.2021.09.183.
[41] S. Hoshian, E. Kankuri, R. H. A. Ras, S. Franssila, V. Jokinen, Water and Blood Repellent Flexible Tubes, Sci. Rep. 7 (2017) 1-8. https://doi.org/10.1038/s41598-017-16369-3.
[42] S. Movafaghi, V. Leszczak, W. Wang, J. A. Sorkin, L. P. Dasi, K. C. Popat, A. K. Kota, Hemocompatibility of Superhemophobic Titania Surfaces, Adv. Healthc. Mater. 6 (2017) 1600717. https://doi.org/10.1002/adhm.201600717.
[43] G. B. Hwang, K. Page, A. Patir, S. P. Nair, E. Allan, I. P. Parkin, The Anti-Biofouling Properties of Superhydrophobic Surfaces are Short-Lived, ACS Nano. 12 (2018) 6050-6058. https://doi.org/10.1021/acsnano.8b02293.
[44] G. M. Halpenny, P. K. Mascharak, Emerging Antimicrobial Applications of Nitric Oxide (NO) and NO-Releasing Materials, Antiinfect. Agents Med. Chem. 9 (2010) 187-197. https://doi.org/10.2174/187152110794785086.
[45] F. C. Fang, Mechanisms of nitric oxide-related antimicrobial activity, J. Clin. Invest. 99 (1997) 2818-2825. https://doi.org/10.1172/jci119473.
[46] E. Culotta, D. E. Koshland, NO news is good news, Science (80-.). 258 (1992) 1862-1865. https://doi.org/10.1126/science. 1361684.
[47] A. Mondal, R. Devine, L. Estes, J. Manuel, P. Singha, J. Mancha, M. Palmer, H. Handa, Highly hydrophobic polytetrafluoroethylene particle immobilization via polydopamine anchor layer on nitric oxide releasing polymer for biomedical applications, J. Colloid Interface Sci. 585 (2021). https://doi.org/10.1016/j.jcis.2020.10.051.
[48] L. M. Estes, P. Singha, S. Singh, T. S. Sakthivel, M. Garren, R. Devine, E. J. Brisbois, S. Seal, H. Handa, Characterization of a nitric oxide (NO) donor molecule and cerium oxide nanoparticle (CNP) interactions and their synergistic antimicrobial potential for biomedical applications, J. Colloid Interface Sci. 586 (2021). https://doi.org/10.1016/j.jcis.2020.10.081.
[49] A. Mondal, P. Singha, M. Douglass, L. Estes, M. Garren, L. Griffin, A. Kumar, H. Handa, A Synergistic New Approach Toward Enhanced Antibacterial Efficacy via Antimicrobial Peptide Immobilization on a Nitric Oxide-Releasing Surface, ACS Appl. Mater. Interfaces. 13 (2021). https://doi.org/10.1021/acsami.1c08921.
[50] M. Garren, P. Maffe, A. Melvin, L. Griffin, S. Wilson, M. Douglass, M. Reynolds, H. Handa, Surface-Catalyzed Nitric Oxide Release via a Metal Organic Framework Enhances Antibacterial Surface Effects, ACS Appl. Mater. Interfaces. 13 (2021). https://doi.org/10.1021/acsami. 1c17248.
[51] M. J. Goudie, J. Pant, H. Handa, Liquid-infused nitric oxide-releasing (LINORel) silicone for decreased fouling, thrombosis, and infection of medical devices, Sci. Rep. 7 (2017) 13623. https://doi.org/10.1038/s41598-017-14012-9.
[52] W. L. Storm, J. Youn, K. P. Reighard, B. V. Worley, H. M. Lodaya, J. H. Shin, M. H. Schoenfisch, Superhydrophobic nitric oxide-releasing xerogels, Acta Biomater. 10 (2014). https://doi.org/10.1016/j.actbio.2014.04.029.
[53] R. A. González-Fuenzalida, Y. Moliner-Martínez, M. González-Béjar, C. Molins-Legua, J. Verdú-Andres, J. Pérez-Prieto, P. Campins-Falcó, In situ colorimetric quantification of silver cations in the presence of silver nanoparticles, Anal. Chem. 85 (2013). https://doi.org/10.1021/ac402822d.
[54] S. S. Latthe, R. S. Sutar, A. K. Bhosale, S. Nagappan, C. S. Ha, K. K. Sadasivuni, S. Liu, R. Xing, Recent developments in air-trapped superhydrophobic and liquid-infused slippery surfaces for anti-icing application, Prog. Org. Coatings. 137 (2019). https://doi.org/10.1016/j.porgcoat.2019.105373.
[55] J. Marlena, J. K. S. Tan, Z. Lin, D. X. Li, B. Zhao, H. L. Leo, S. Kim, C. H. Yap, Monolithic polymeric porous superhydrophobic material with pneumatic plastron stabilization for functionally durable drag reduction in blood-contacting biomedical applications, NPG Asia Mater. 13 (2021). https://doi.org/10.1038/s41427-021-00325-9.
[56] Y. Tokudome, K. Okada, A. Nakahira, M. Takahashi, Switchable and reversible water adhesion on superhydrophobic titanate nanostructures fabricated on soft substrates: Photopatternable wettability and thermomodulatable adhesivity, J. Mater. Chem. A. 2 (2014). https://doi.org/10.1039/c3ta13536e.
[57] S. Musić, N. Filipović-Vinceković, L. Sekovanić, Precipitation of amorphous SiO2 particles and their properties, Brazilian J. Chem. Eng. 28 (2011). https://doi.org/10.1590/S0104-66322011000100011.
[58] R. Nazar, M. Sangermano, A. Vitale, R. Bongiovanni, Silver polymer nanocomposites by photoreduction of AgNO3 and simultaneous photocrosslinking of the acrylic matrix: Effect of PVP on Ag particle formation, J. Polym. Eng. 38 (2018). https://doi.org/10.1515/polyeng-2017-0156.
[59] R. Devine, M. Douglass, M. Ashcraft, N. Tayag, H. Handa, Development of Novel Amphotericin B-Immobilized Nitric Oxide-Releasing Platform for the Prevention of Broad-Spectrum Infections and Thrombosis, ACS Appl. Mater. Interfaces. 13 (2021). https://doi.org/10.1021/acsami.1c01330.
[60] M. J. Goudie, J. Pant, H. Handa, Liquid-infused nitric oxide-releasing (LINORel) silicone for decreased fouling, thrombosis, and infection of medical devices, Sci. Rep. 7 (2017) 13623. https://doi.org/10.1038/s41598-017-14012-9.
[61] N. Barraud, M. Kelso, S. Rice, S. Kjelleberg, Nitric Oxide: A Key Mediator of Biofilm Dispersal with Applications in Infectious Diseases, Curr. Pharm. Des. 21 (2014). https://doi.org/10.2174/1381612820666140905112822.
[62] M. W. Vaughn, L. Kuo, J. C. Liao, Estimation of nitric oxide production and reactionrates in tissue by use of a mathematical model, Am. J. Physiol. Circ. Physiol. 274 (1998) H2163-H2176. https://doi.org/10.1152/ajpheart. 1998.274.6.H2163.
[63] B. McEvoy, N. J. Rowan, Terminal sterilization of medical devices using vaporized hydrogen peroxide: a review of current methods and emerging opportunities, J. Appl. Microbiol. 127 (2019). https://doi.org/10.1111/jam.14412.
[64] M. A. De Groote, F. C. Fang, NO inhibitions: Antimicrobial properties of nitric oxide, Clin. Infect. Dis. 21 (1995). https://doi.org/10.1093/clinids/21.Supplement_2.S162.
[65] F. C. Fang, Perspectives series: host/pathogen interactions. Mechanisms of nitric oxide-related antimicrobial activity, J. Clin. Invest. 99 (1997) 2818-2825. https://doi.org/10.1172/JCI119473.
[66] W. R. Li, X. B. Xie, Q. S. Shi, H. Y. Zeng, Y. S. Ou-Yang, Y. Ben Chen, Antibacterial activity and mechanism of silver nanoparticles on Escherichia coli, Appl. Microbiol. Biotechnol. 85 (2010). https://doi.org/10.1007/s00253-009-2159-5.
[67] N. Durán, M. Durán, M. B. de Jesus, A. B. Seabra, W. J. Fávaro, G. Nakazato, Silver nanoparticles: A new view on mechanistic aspects on antimicrobial activity, Nanomedicine Nanotechnology, Biol. Med. 12 (2016). https://doi.org/10.1016/j.nano.2015.11.016.
[68] A. L. Urzedo, M. C. Gonçalves, M. H. M. Nascimento, C. B. Lombello, G. Nakazato, A. B. Seabra, Multifunctional alginate nanoparticles containing nitric oxide donor and silver nanoparticles for biomedical applications, Mater. Sci. Eng. C. 112 (2020). https://doi.org/10.1016/j.msec.2020.110933.
[69] W. R. Rolim, J. C. Pieretti, D. L. S. Reno, B. A. Lima, M. H. M. Nascimento, F. N. Ambrosio, C. B. Lombello, M. Brocchi, A. C. S. De Souza, A. B. Seabra, Antimicrobial Activity and Cytotoxicity to Tumor Cells of Nitric Oxide Donor and Silver Nanoparticles Containing PVA/PEG Films for Topical Applications, ACS Appl. Mater. Interfaces. (2019). https://doi.org/10.1021/acsami.8b19021.
[70] E. Fadeeva, V. K. Truong, M. Stiesch, B. N. Chichkov, R. J. Crawford, J. Wang, E. P. Ivanova, Bacterial retention on superhydrophobic titanium surfaces fabricated by femtosecond laser ablation, Langmuir. 27 (2011)3012-9. https://doi.org/10.1021/la104607g.
[71] S. Zheng, M. Bawazir, A. Dhall, H. E. Kim, L. He, J. Heo, G. Hwang, Implication of Surface Properties, Bacterial Motility, and Hydrodynamic Conditions on Bacterial Surface Sensing and Their Initial Adhesion, Front. Bioeng. Biotechnol. 9 (2021). https://doi.org/10.3389/fbioe.2021.643722.
[72] N. Mackman, Triggers, targets and treatments for thrombosis, Nature. 451 (2008). https://doi.org/10.1038/nature06797.
[73] U. T. Seyfert, V. Biehl, J. Schenk, In vitro hemocompatibility testing of biomaterials according to the ISO 10993-4, in: Biomol. Eng., 2002. https://doi.org/10.1016/S1389-0344 (02) 00015-1.
[74] C. A. Dos Santos, M. M. Seckler, A. P. Ingle, I. Gupta, S. Galdiero, M. Galdiero, A. Gade, M. Rai, Silver nanoparticles: Therapeutical uses, toxicity, and safety issues, J. Pharm. Sci. 103 (2014). https://doi.org/10.1002/jps.24001.
[75] ISO 10993-5, Biological evaluation of medical devices. Part 5: Tests for in vitro cytotocity, 2009.
[76] S. Arora, J. Jain, J. M. Rajwade, K. M. Paknikar, Interactions of silver nanoparticles with primary mouse fibroblasts and liver cells, Toxicol. Appl. Pharmacol. 236 (2009). https://doi.org/10.1016/j.taap.2009.02.020.

1. A coating composition comprising
(a) silicon oxide nanoparticles, wherein a perfluorolkylsiloxane is covalently bonded to the silicon oxide nanoparticles;
(b) silver nanoparticles, wherein a perfluorolkylsiloxane is covalently bonded to the silver nanoparticles; and
(c) a solvent.

2. The composition of claim 1, wherein the silicon oxide nanoparticles have an average particle size of from about 5 nm to about 50 nm and the silver nanoparticles have an average particle size of from about 5 nm to about 100 nm.

3. (canceled)

4. The composition of claim 1, wherein the weight ratio of the silicon oxide nanoparticles to the silver nanoparticles is from 0.1 to 5 to 5:0.1.

5. The composition of claim 1, wherein the perfluorolkylsiloxane has the formula R²—Si(OR¹)₃, wherein R¹ is a substituted or unsubstituted C₁-C₂₀ alkyl group, and R² is a C₁-C₂₀ perfluoroalkyl group.

6. (canceled)

7. (canceled)

8. The composition of claim 5, wherein each R¹ is methyl or ethyl, and R² is a C₈ perfluoroalkyl group.

9. The composition of claim 1, wherein the silicon oxide nanoparticles and the silver nanoparticles are from 1 weight percent to about 20 weight percent of the composition.

10. (canceled)

11. The composition of claim 1, wherein the organic solvent comprises a C₁ to C₁₀ hydrocarbon.

12. The composition of claim 1, wherein the composition further comprises an elastomeric polymer, wherein the elastomeric polymer comprises one or more of a copolyester elastomer, a polyether block amide elastomer, a polyurethane elastomer, a polyolefin based-copolymer elastomer, a styrenic copolymer elastomer, an ionomer elastomer, or any combination thereof.

13. (canceled)

14. (canceled)

15. The composition of claim 1, wherein the composition further comprises a silicone elastomer and a curing agent.

16. The composition of claim 15, wherein a weight ratio of the silicon oxide nanoparticles and the silver nanoparticles to the silicone elastomer and a curing agent is from 0.1 to 5 to 5:0.1.

17-26. (canceled)

27. A coated article comprising a coating on at least one surface of the article, wherein the coating comprises
(a) silicon oxide nanoparticles, wherein a perfluorolkylsiloxane is covalently bonded to the silicon oxide nanoparticles;
(b) silver nanoparticles, wherein a perfluorolkylsiloxane is covalently bonded to the silver nanoparticles; and
(c) a nitric oxide releasing compound.

28. The coated article of claim 27, wherein the silicon oxide nanoparticles have an average particle size of from about 5 nm to about 50 nm and the silver nanoparticles have an average particle size of from about 5 nm to about 100 nm.

29. (canceled)

30. (canceled)

31. The coated article of claim 27, wherein the perfluorolkylsiloxane has the formula R²—Si(OR¹)₃, wherein R¹ is a substituted or unsubstituted C₁-C₂₀ alkyl group, and R² is a C₁-C₂₀ perfluoroalkyl group.

32. (canceled)

33. (canceled)

34. The coated article of claim 31, wherein each R¹ is methyl or ethyl, and R² is a C₈ perfluoroalkyl group.

35. The coated article of claim 27, wherein the composition coating further comprises a silicone elastomer.

36. The coated article of claim 35, wherein the silicone elastomer is from 1 weight percent to about 20 weight percent of the coating and the weight ratio of the silicon oxide nanoparticles and the silver nanoparticles to the silicone elastomer is from 0.1 to 5 to 5:0.1.

37. (canceled)

38. The coated article of claim 27, wherein the nitric oxide releasing compound is a S-nitrosothiol compound.

39. The coated article of claim 27, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, or any combination thereof.

40. The coated article of claim 27, wherein the coating further comprises an elastomeric polymer, wherein the elastomeric polymer comprises one or more of a copolyester elastomer, a polyether block amide elastomer, a polyurethane elastomer, a polyolefin based-copolymer elastomer, a styrenic copolymer elastomer, an ionomer elastomer, or any combination thereof.

41. (canceled)

42. (canceled)

43. (canceled)

44. The coated article of claim 27, wherein the article comprises a polymeric grade material, a medical device, a surface or article in a hospital or medical facility, or a surface in an automobile, boat, or aircraft.

45-57. (canceled)
